(19) United States
(12) Patent Application Publication
(10) Pub. No.: US 2007/0083945 A1
(43) Pub. Date: Apr. 12, 2007

(54) Nucleic acid molecules and other molecules associated with plants

(76) Inventors: Joseph R. Byrum, Des Moines, IA (US); Conrad H. Halling, St. Louis, MO (US); David K. Kovalic, St. Louis, MO (US)

Correspondence Address: ARNOLD & PORTER, LLP, 555 TWELFTH STREET, N.W., ATTN: IP DOCKETING, WASHINGTON, DC, 20004, US

(21) Appl. No.: 11/497,489

(22) Filed: Aug. 2, 2006

Related U.S. Application Data
(63) Continuation of application No. 09/521,640, filed on Mar. 10, 2000, now abandoned.

(51) Int. Cl. A01H 1/00; C07K 14/415; C12N 15/87; C07H 21/04

(52) U.S. Cl. 800/278; 536/23.6; 530/370

(57) ABSTRACT

The present invention is in the field of plant genetics. More specifically the invention relates to nucleic acid molecules and nucleic acid molecules that contain markers, in particular, single nucleotide polymorphism (SNP) and repetitive element markers. In addition, the present invention provides nucleic acid molecules having regulatory elements or encoding proteins or fragments thereof. The invention also relates to proteins and fragments of proteins so encoded and antibodies capable of binding the proteins. The invention also relates to methods of using the nucleic acid molecules, markers, repetitive elements and fragments of repetitive elements, regulatory elements, proteins and fragments of proteins.

### CROSS REFERENCE TO RELATED APPLICATION

This application is a continuation of U.S. application Ser. No. 09/521,640 filed Mar. 10, 2000, which is herein incorporated by reference in its entirety.

### INCORPORATION OF SEQUENCE LISTING

This application contains a sequence listing, which is contained on three identical CD-ROMs: two copies of the sequence listing (Copy 1 and Copy 2) and a sequence listing Computer Readable Form (CRF), all of which are herein incorporated by reference. All three sequence listing CD-ROMs each contain one file called “15750E seq list.rpt” which is 190,044,160 bytes in size (measured in Windows XP) and which was created on Aug. 2, 2006.

### FIELD OF THE INVENTION

The present invention is in the field of plant genetics. More specifically the invention relates to nucleic acid molecules and nucleic acid molecules that contain markers, in particular, single nucleotide polymorphism (SNP) and repetitive element markers. In addition, the present invention provides nucleic acid molecules having regulatory elements or encoding proteins or fragments thereof. The invention also relates to proteins and fragments of proteins so encoded and antibodies capable of binding the proteins. The invention also relates to methods of using the nucleic acid molecules, markers, repetitive elements and fragments of repetitive elements, regulatory elements, proteins and fragments of proteins.

### BACKGROUND OF THE INVENTION

I. Sequence Tagged Connector Nucleic Acid Molecules and the Bacterial Artificial Chromosomes (BACs) Containing these Sequences.

Sequence tagged connectors, or STCs, are sequences of insert data generated from both ends (at the vector-insert point) of a BAC clone in a genomic library. These sequences, and BACs containing these STC sequences, can be used, for example, for marker development, genetic mapping or linkage analysis, marker assisted breeding, and physical genome mapping (Venter, et al., Nature, 381:364-366 (1996), the entirety of which is herein incorporated by reference; Choi and Wing, on the Worldwide web at genome.clemson.edu/protocols2-nj.html July, 1998). STCs can represent a copy of up to a full length of a mRNA transcript, a promoter element or part of a promoter, can contain simple sequence repeats (also called microsatellites) repetitive elements or fragments of repetitive elements, other DNA markers, or any combination thereof.

Markers have been used in genetic mapping which can be a step in isolating a gene. Genetic mapping or linkage analysis is based on the level at which markers and genes are co-inherited (Rothwell, Understanding Genetics. 4^{th} Ed., Oxford University Press, New York, p. 703 (1988). Statistical tests like chi-square analysis can be used to test the randomness of segregation or linkage (Kochert, The Rockefeller Foundation International Program on Rice Biotechnology, University of Georgia, Athens, Ga., pp 1-14 (1989), the entirety of which is herein incorporated by reference. In linkage mapping, the proportion of recombinant individuals out of the total mapping population provides the information for determining the genetic distance between the loci (Young, Encyclopedia of Agricultural Science, Vol. 3, pp 275-282 (1994), the entirety of which is herein incorporated by reference).

Classical mapping studies utilize easily observable, visible traits instead of molecular markers. These visible traits are also known as naked eye polymorphisms. These traits can be morphological like plant height, fruit size, shape and color or physiological like disease response, photoperiod sensitivity or crop maturity. Visible traits are useful and are still in use because they represent actual phenotypes and are easy to score without any specialized lab equipment. By contrast, the other types of genetic markers are arbitrary loci for use in linkage mapping and often not associated to specific plant phenotypes (Young, Encyclopedia of Agricultural Science, Vol. 3, pp. 275-282 (1994). Many morphological markers cause such large effects on phenotype that they are undesirable in breeding programs. Many other visible traits have the disadvantage of being developmentally regulated (i.e., expressed only at certain stages; or in specific tissues and organs). Often times, visible traits mask the effects of linked minor genes making it nearly impossible to identify desirable linkages for selection (Tanksely, et al., Biotech. 7:257-264 (1989), the entirety of which is herein incorporated by reference).

Although a number of important agronomic characters are controlled by loci having major effects on phenotype, many economically important traits, such as yield and some forms of disease resistance, are quantitative in nature. This type of phenotypic variation in a trait is characterized by continuous, normal distribution of phenotypic values in a particular population (Beckmann and Soller, Oxford Surveys of Plant Molecular Biology, Miffen. (ed.), Vol. 3, Oxford University Press, UK., pp. 196-250 (1986), the entirety of which is herein incorporated by reference). Such traits are governed by a large number of loci, Quantitative Trait Loci (QTL), each of which can make a small positive or negative effect to the final phenotype value of the trait (Beckmann and Soller, Oxford Surveys of Plant Molecular Biology, Miffen. (ed.), Vol. 3, Oxford University Press, U.K., pp. 196-250 (1986). Loci contributing to such genetic variation are often; termed minor genes as opposed to major genes with large effects that follow a Mendelian pattern of inheritance. Polygenic traits are also predicted to follow a Mendelian type of inheritance, however the contribution of each locus is expressed as an increase or decrease in the final trait value.

Markers have been used in physical mapping studies with BAC libraries made from plant genomes. Such mapping studies have been carried out in rice (Kim et al., Genomics 34:213-218 (1996), herein incorporated by reference; Hang, Plant Mol. Biol. 35:129-133 (1997), herein incorporated by reference; Zhang and Wing., Plant Mol. Bio. 35:115-127 (1997) herein incorporated by reference; Chen et al., Proc. Acad. Sci. (U.S.A.) 94:3431-3435 (1997) herein incorporated by reference; Wang et al., Plant J. 7:525-533 (1995) herein incorporated by reference) sorghum (Zwick et al., Genetics 148:1983-1992 (1998) herein incorporated by reference; Zhang, et al., Molecular Breeding 2:11-24 (1996) the entirety of which is herein incorporated by reference) maize, (Chen, et al., Proc. Acad. Sci. (U.S.A.) 94:3431-3435 (1997), and Arabidopsis (Kim, et al., Genomics 34:213-218 (1996) the entirety of which is herein incorporated by reference.

Repetitive elements have been used in physical mapping in cereals (Ananiev, et al., Proc. Acad. Sci. (U.S.A.) 95:13073-8 (1998), the entirety of which is herein incorporated by reference; McLean et al., Mol Gen Genet 253:687-694 (1997), the entirety of which is herein incorporated by reference).

II. Sequence Comparisions

STCs and sequenced BACs can be compared, for example, to sequences that encode promoters or proteins. These homologies can be determined by similarity searches (Adams, et al., Science 252:1651-1656 (1991), herein incorporated by reference).

A characteristic feature of a DNA sequence is that it can be compared with other DNA sequences. Sequence comparisons can be undertaken by determining the similarity of the test or query sequence with sequences in publicly available or propriety databases (“similarity analysis”) or by searching for certain motifs (“intrinsic sequence analysis”) (e.g., cis elements) (Coulson, Trends in Biotechnology, 12:76-80 (1994), the entirety of which is herein incorporated by reference; Birren, et al., Genome Analysis, 1:543-559 (1997), the entirety of which is herein incorporated by reference).

Similarity analysis includes database search and alignment. Examples of public databases include the DNA Database of Japan (DDBJ) (on the Worldwide web at ddbj.nig.ac.jp/); Genebank (on the Worldwide web at ncbi.nlm.nih.gov/web/Genbank/Index.htlm); and the European Molecular Biology Laboratory Nucleic Acid Sequence Database (EMBL) (on the Worldwide web at ebi.ac.uk/ebi_docs/embl_db.html). A number of different search algorithms have been developed, one example of which are the suite of programs referred to as BLAST programs. There are five implementations of BLAST, three designed for nucleotide sequences queries (BLASTN, BLASTX, and TBLASTX) and two designed for protein sequence queries (BLASTP and TBLASTN) (Coulson, Trends in Biotechnology, 12:76-80 (1994); Birren, et al., Genome Analysis, 1:543-559 (1997)).

BLASTN takes a nucleotide sequence (the query sequence) and its reverse complement and searches them against a nucleotide sequence database. BLASTN was designed for speed, not maximum sensitivity, and may not find distantly related coding sequences. BLASTX takes a nucleotide sequence, translates it in three forward reading frames and three reverse complement reading frames, and then compares the six translations against a protein sequence database. BLASTX is useful for sensitive analysis of preliminary (single-pass) sequence data and is tolerant of sequencing errors (Gish and States, Nature Genetics, 3:266-272 (1993), the entirety of which is herein incorporated by reference). BLASTN and BLASTX may be used in concert for analyzing STC data (Coulson, Trends in Biotechnology, 12:76-80 (1994); Birren, et al., Genome Analysis, 1:543-559(1997).

Given a coding nucleotide sequence and the protein it encodes, it is often preferable to use the protein as the query sequence to search a database because of the greatly increased sensitivity to detect more subtle relationships. This is due to the larger alphabet of proteins (20 amino acids) compared with the alphabet of nucleic acid sequences (4 bases), where it is far easier to obtain a match by chance. In addition, with nucleotide alignments, only a match (positive score) or a mismatch (negative score) is obtained, but with proteins, the presence of conservative amino acid substitutions can be taken into account. Here, a mismatch may yield a positive score if the non-identical residue has physical/chemical properties similar to the one it replaced. Various scoring matrices are used to supply the substitution scores of all possible amino acid pairs. A general purpose scoring system is the BLOSUM62 matrix (Henikoff and Henikoff, Proteins, 17:49-61 (1993), the entirety of which is herein incorporated by reference), which is currently the default choice for BLAST programs. BLOSUM62 is tailored for alignments of moderately diverged sequences and thus may not yield the best results under all conditions. Altschul, J. Mol. Biol. 36:290-300 (1993), the entirety of which is herein incorporated by reference, uses a combination of three matrices to cover all contingencies. This may improve sensitivity, but at the expense of slower searches. In practice, a single BLOSUM62 matrix is often used but others (PAM40 and PAM250) may be attempted when additional analysis is necessary. Low PAM matrices are directed at detecting very strong but localized sequence similarities, whereas high PAM matrices are directed at detecting long but weak alignments between very distantly related sequences.

Homologues in other organisms are available that can be used for comparative sequence analysis. Multiple alignments are performed to study similarities and differences in a group of related sequences. CLUSTAL W is a multiple sequence alignment package available that performs progressive multiple sequence alignments based on the method of Feng and Doolittle, J. Mol. Evol. 25:351-360 (1987), the entirety of which is herein incorporated by reference. Each pair of sequences is aligned and the distance between each pair is calculated; from this distance matrix, a guide tree is calculated, and all of the sequences are progressively aligned based on this tree. A feature of the program is its sensitivity to the effect of gaps on the alignment; gap penalties are varied to encourage the insertion of gaps in probable loop regions instead of in the middle of structured regions. Users can specify gap penalties, choose between a number of scoring matrices, or supply their own scoring matrix for both the pairwise alignments and the multiple alignments. CLUSTAL W for UNIX and VMS systems is available by anonymous ftp at ebi.ac.uk. Another program is MACAW (Schuler et al., Proteins, Struct. Func. Genet, 9:180-190 (1991), the entirety of which is herein incorporated by reference, for which both Macintosh and Microsoft Windows versions are available. MACAW uses a graphical interface, provides a choice of several alignment algorithms, and is available by anonymous ftp at: ncbi.nlm.nih.gov (directory/pub/macaw).

Sequence motifs are derived from multiple alignments and can be used to examine individual sequences or an entire database for subtle patterns. With motifs, it is sometimes possible to detect distant relationships that may not be demonstrable based on comparisons of primary sequences alone. Currently, the largest collection of sequence motifs in the world is PROSITE (Bairoch and Bucher, Nucleic Acid Research, 22:3583-3589 (1994), the entirety of which is herein incorporated by reference). PROSITE may be accessed via either the ExPASy server on the World Wide Web or anonymous ftp site. Many commercial sequence analysis packages also provide search programs that use PROSITE data.

A resource for searching protein motifs is the BLOCKS E-mail server developed by S. Henikoff, Trends Biochem Sci., 18:267-268 (1993), the entirety of which is herein incorporated by reference; Henikoff and Henikoff, Nucleic Acid Research, 19:6565-6572 (1991), the entirety of which is herein incorporated by reference; Henikoff and Henikoff, Proteins, 17:49-61 (1993). BLOCKS searches a protein or nucleotide sequence against a database of protein motifs or “blocks.” Blocks are defined as short, ungapped multiple alignments that represent highly conserved protein patterns. The blocks themselves are derived from entries in PROSITE as well as other sources. Either a protein or nucleotide query can be submitted to the BLOCKS server; if a nucleotide sequence is submitted, the sequence is translated in all six reading frames and motifs are sought in these conceptual translations. Once the search is completed, the server will return a ranked list of significant matches, along with an alignment of the query sequence to the matched BLOCKS entries.

Conserved protein domains can be represented by two-dimensional matrices, which measure either the frequency or probability of the occurrences of each amino acid residue and deletions or insertions in each position of the domain. This type of model, when used to search against protein databases, is sensitive and usually yields more accurate results than simple motif searches. Two popular implementations of this approach are profile searches (such as GCG program ProfileSearch) and Hidden Markov Models (HMMs) (Krough, etal., J. Mol. Biol. 235:1501-1531 (1994); Eddy, Current Opinion in Structural Biology 6:361-365 (1996), both of which are herein incorporated by reference in their entirety). In both cases, a large number of common protein domains have been converted into profiles, as present in the PROSITE library, or HHM models, as in the Pfam protein domain library (Sonnhammer, et al., Proteins 28:405-420 (1997), the entirety of which is herein incorporated by reference). Pfam contains more than 500 HMM models for enzymes, transcription factors, signal transduction molecules, and structural proteins. Protein databases can be queried with these profiles or HMM models, which will identify proteins containing the domain of interest. For example, HMMSW or HMMFS, two programs in a public domain package called HMMER (Sonnhammer, et al., Proteins 28:405-420 (1997)) can be used.

PROSITE and BLOCKS represent collected families of protein motifs. Thus, searching these databases entails submitting a single sequence to determine whether or not that sequence is similar to the members of an established family. Programs working in the opposite direction compare a collection of sequences with individual entries in the protein databases. An example of such a program is the Motif Search Tool, or MoST (Tatusov, et al., Proc. Natl. Acad. Sci. 91:12091-12095 (1994), the entirety of which is herein incorporated by reference). On the basis of an aligned set of input sequences, a weight matrix is calculated by using one of four methods (selected by the user); a weight matrix is simply a representation, position by position in an alignment, of how likely a particular amino acid will appear. The calculated weight matrix is then used to search the databases. To increase sensitivity, newly found sequences are added to the original data set, the weight matrix is recalculated, and the search is performed again. This procedure continues until no new sequences are found.

### SUMMARY OF THE INVENTION

The present invention includes and provides a substantially purified nucleic acid molecule, the nucleic acid molecule capable of specifically hybridizing to a second nucleic acid molecule having a nucleic acid sequence selected from the group consisting of SEQ ID NO: 1 through SEQ ID NO: 304905 or complement or fragment thereof.

The present invention provides a substantially purified nucleic acid molecule comprising a nucleic acid molecule or fragment thereof having a pair of defined ends, wherein the pair of defined ends are selected from the defined ends in Table A of U.S. application Ser. No. 09/521,640, which is herein incorporated by reference in its entirety.

The present invention provides a substantially purified protein or fragment thereof encoded by a first nucleic acid molecule which specifically hybridizes to a second nucleic acid molecule, the second nucleic acid molecule having a nucleic acid sequence selected from the group consisting of SEQ ID NO:1 through SEQ ID NO:304905 or complements thereof.

The present invention provides a substantially purified protein or fragment thereof encoded by a nucleic acid sequence selected from the group consisting of SEQ ID NO:1 through SEQ ID NO:304905 or complements thereof.

The present invention provides a transformed plant having a nucleic acid molecule which comprises: (A) an exogenous promoter region which functions in a plant cell to cause the production of a mRNA molecule; which is linked to (B) a structural nucleic acid molecule, wherein the structural nucleic acid molecule is selected from the group consisting of SEQ ID NO:1 through SEQ ID NO:304905 or complements thereof or fragments of either; which is linked to (C) a 3′ non-translated sequence that functions in a plant cell to cause termination of transcription and addition of polyadenylated ribonucleotides to a 3′ end of the mRNA molecule.

The present invention provides a transformed plant having a nucleic acid molecule which comprises: (A) an exogenous promoter region which functions in a plant cell to cause the production of a mRNA molecule wherein the promoter nucleic acid molecule is selected from the group consisting of SEQ ID NO:1 through SEQ ID NO:304905 or complements thereof or fragments of either; which is linked to (B) a structural nucleic acid molecule encoding a protein or peptide; which is linked to (C) a 3′ non-translated sequence that functions in a plant cell to cause termination of transcription and addition of polyadenylated ribonucleotides to a 3′ end of the mRNA molecule.

The present invention provides a transformed plant having a nucleic acid molecule which comprises: (A) an exogenous promoter region which functions in a plant cell to cause the production of a mRNA molecule; which is linked to (B) a transcribed nucleic acid molecule with a transcribed strand and a non-transcribed strand, wherein the transcribed strand is complementary to a nucleic acid molecule having a nucleic acid sequence selected from the group consisting of SEQ ID NO:1 through SEQ ID NO:304905 or complements thereof or fragments of either and the transcribed strand is complementary to an endogenous mRNA molecule; which is linked to (C) a 3′ non-translated sequence that functions in plant cells to cause termination of transcription and addition of polyadenylated ribonucleotides to a 3′ end of the mRNA molecule.

The present invention provides a transformed plant having a nucleic acid molecule which comprises: (A) an exogenous promoter region which functions in a plant cell to cause the production of a mRNA molecule wherein the promoter nucleic acid molecule is selected from the group consisting of SEQ ID NO:1 through SEQ ID NO:304905 or complements thereof or fragments of either; which is linked to (B) a transcribed nucleic acid molecule with a transcribed strand and a non-transcribed strand, wherein the transcribed strand is complementary to an endogenous mRNA molecule; which is linked to (C) a 3′ non-translated sequence that functions in plant cells to cause termination of transcription and addition of polyadenylated ribonucleotides to a 3′ end of the mRNA molecule.

The present invention provides a computer readable medium having recorded thereon one or more of the nucleotide sequences depicted in SEQ ID NO:1 through SEQ ID NO: 304905.

The present invention provides a method of introgressing a trait into a plant comprising using a nucleic acid marker for marker assisted selection of the plant, the nucleic acid marker complementary to a nucleic acid sequence selected from the group consisting of SEQ ID NO: 1 through SEQ ID NO: 304905 or complements thereof, and introgressing the trait into a plant.

The present invention provides a method for screening for a trait comprising interrogating genomic DNA for the presence or absence of a marker molecule that is genetically linked to a nucleic acid sequence complementary to a nucleic acid sequence selected from the group consisting of SEQ ID NO: 1 through SEQ ID NO: 304905 or complements thereof; and detecting the presence or absence of the marker.

The present invention provides a method for determining the likelihood of the level, presence or absence of a trait in a plant comprising the steps of: (A) obtaining genomic DNA from the plant; (B) detecting a marker nucleic acid molecule; the marker nucleic acid molecule wherein the marker nucleic acid molecule specifically hybridizes with a nucleic acid sequence that is genetically linked to a nucleic acid sequence complementary to a nucleic acid sequence selected from the group consisting of SEQ ID NO: 1 through SEQ ID NO: 304905 or complements thereof; (C) and determining the level, presence or absence of the marker nucleic acid molecule, wherein the level, presence or absence of the marker nucleic acid molecule is indicative of the likely presence in the plant of the trait.

The present invention provides a method for determining a genomic polymorphism in a plant that is predictive of a trait comprising the steps: (A) incubating a marker nucleic acid molecule, under conditions permitting nucleic acid hybridization, and a complementary nucleic acid molecule obtained from the plant, the marker nucleic acid molecule having a nucleic acid sequence selected from the group consisting of SEQ ID NO: 1 through SEQ ID NO: 304905 or complements thereof; (B) permitting hybridization between the marker nucleic acid molecule and the complementary nucleic acid molecule obtained from the plant; and (C) detecting the presence of the polymorphism.

The present invention provides a method of determining an association between a polymorphism and a plant trait comprising: (A) hybridizing a nucleic acid molecule specific for the polymorphism to genetic material of a plant, wherein the nucleic acid molecule comprises a nucleic acid sequence selected from the group consisting of SEQ ID NO: 1 through SEQ ID NO: 304905 or complements thereof; and (B) calculating the degree of association between the polymorphism and the plant trait.

### DETAILED DESCRIPTION OF THE INVENTION

Agents of the Invention:

(a) Nucleic Acid Molecules

Agents of the present invention include nucleic acid molecules and more specifically BACs and STC nucleic acid molecules or fragments thereof

A subset of the nucleic acid molecules of the present invention includes nucleic acid molecules that are marker molecules. Another subset of the nucleic molecules of the present invention include nucleic acid molecules that are promoters and/or regulatory elements. Another subset of the nucleic acid molecules of the present invention include nucleic acid molecules that encode proteins or fragments of proteins. In a preferred embodiment the nucleic acid molecules of the present invention are derived from Glycine max (soybean) and more preferably Glycine max, genotype A3244.

Fragment STC nucleic acid molecules and fragments of BACs may encode significant portion(s) of, or indeed most of, the STC or BAC nucleic acid molecule. In addition, a fragment nucleic acid molecule can encode a Glycine max protein or fragment thereof. Alternatively, the fragments may comprise smaller oligonucleotides (having from about 15 to about 250 nucleotide residues, and more preferably, about 15 to about 30 nucleotide residues).

The term “substantially purified”, as used herein, refers to a molecule separated from substantially all other molecules normally associated with it in its native state. More preferably a substantially purified molecule is the predominant species present in a preparation. A substantially purified molecule may be greater than 60% free, preferably 75% free, more preferably 90% free, and most preferably 95% free from the other molecules (exclusive of solvent) present in the natural mixture. The term “substantially purified” is not intended to encompass molecules present in their native state.

The agents of the present invention will preferably be “biologically active” with respect to either a structural attribute, such as the capacity of a nucleic acid to hybridize to another nucleic acid molecule, or the ability of a protein to be bound by an antibody (or to compete with another molecule for such binding). Alternatively, such an attribute may be catalytic, and thus involve the capacity of the agent to mediate a chemical reaction or response.

The agents of the present invention may also be recombinant. As used herein, the term recombinant means any agent (e.g., DNA, peptide etc.), that is, or results, however indirect, from human manipulation of a nucleic acid molecule.

It is understood that the agents of the present invention may be labeled with reagents that facilitate detection of the agent (e.g., fluorescent labels (Prober, et al., Science 238:336-340 (1987); Albarella et al., EP 144914, chemical labels (Sheldon et al., U.S. Pat. No. 4,582,789; Albarella et al., U.S. Pat. No. 4,563,417, modified bases (Miyoshi et al., EP 119448, all of which are hereby incorporated by reference in their entirety).

It is further understood, that the present invention provides bacterial, viral, microbial, insect, fungal and plant cells comprising the agents of the present invention. The BAC nucleic acid molecules of the present invention include, without limitation, BAC nucleic acid molecules having inserts with two defined ends (STCs) as set forth in Table A of U.S. application Ser. No. 09/521,640, which is herein incorporated by reference in its entirety. It is understood that fragments of such BAC molecules can contain one or neither of the defined ends.

STC nucleic acid molecules or fragment STC nucleic acid molecules, or BACs or fragments thereof, of the present invention are capable of specifically hybridizing to other nucleic acid molecules under certain circumstances. As used herein, two nucleic acid molecules are said to be capable of specifically hybridizing to one another if the two molecules are capable of forming an anti-parallel, double-stranded nucleic acid structure. A nucleic acid molecule is said to be the “complement” of another nucleic acid molecule if they exhibit complete complementarity. As used herein, molecules are said to exhibit “complete complementarity” when every nucleotide of one of the molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are described by Sambrook, et al., Molecular Cloning, A Laboratory Manual, 2nd Ed., Cold Spring Harbor Press, Cold Spring Harbor, N.Y. (1989), and by Haymes, et al, Nucleic Acid Hybridization, A Practical Approach, IRL Press, Washington, D.C. (1985), the entirety of which is herein incorporated by reference. Departures from complete complementarity are therefore permissible, as long as such departures do not completely preclude the capacity of the molecules to form a double-stranded structure. Thus, in order for an STC nucleic acid molecule, fragment STC nucleic acid molecule, BAC nucleic acid molecule or fragment BAC nucleic acid molecule to serve as a primer or probe it need only be sufficiently complementary in sequence to be able to form a stable double-stranded structure under the particular solvent and salt concentrations employed.

Appropriate stringency conditions which promote DNA hybridization are, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

In a preferred embodiment, a nucleic acid of the present invention will specifically hybridize to one or more of the nucleic acid molecules set forth in SEQ ID NO: 1 through SEQ ID NO: 304905 or complements thereof under moderately stringent conditions, for example at about 2.0×SSC and about 40° C.

In a particularly preferred embodiment, a nucleic acid of the present invention will specifically hybridize to one or more of the nucleic acid molecules set forth in SEQ ID NO:1 through SEQ ID NO: 304905 or complements thereof under high stringency conditions. In one aspect of the present invention, the nucleic acid molecules of the present invention have one or more of the nucleic acid sequences set forth in SEQ ID NO: 1 through to SEQ ID NO: 304905 or complements thereof. In another aspect of the present invention, one or more of the nucleic acid molecules of the present invention share between 100% and 90% sequence identity with one or more of the nucleic acid sequences set forth in SEQ ID NO: 1 through to SEQ ID NO: 304905 or complements thereof. In a further aspect of the present invention, one or more of the nucleic acid molecules of the present invention share between 100% and 95% sequence identity with one or more of the nucleic acid sequences set forth in SEQ ID NO: 1 through to SEQ ID NO: 304905 or complements thereof. In a more preferred aspect of the present invention, one or more of the nucleic acid molecules of the present invention share between 100% and 98% sequence identity with one or more of the nucleic acid sequences set forth in SEQ ID NO: 1 through to SEQ ID NO: 304905 or complements thereof. In an even more preferred aspect of the present invention, one or more of the nucleic acid molecules of the present invention share between 100% and 99% sequence identity with one or more of the sequences set forth in SEQ ID NO: 1 through to SEQ ID NO: 304905 or complements thereof. In a further, even more preferred aspect of the present invention, one or more of the nucleic acid molecules of the present invention exhibit 100% sequence identity with one or more nucleic acid molecules present within the genomic library herein designated BAC#1 (Monsanto Company, St. Louis, Mo., United States of America).

It is understood that the present invention encompasses fragments of such nucleic acid molecules and that such nucleic acid fragments may contain one, part of one, or neither of the defined sequences.

(a)(1) Nucleic Acid Molecule Markers

One aspect of the present invention concerns nucleic acid molecules SEQ ID NO:1 through SEQ ID NO:304905 or complements thereof, that contain microsatellites, single nucleotide substitutions (SNPs), repetitive elements or parts of repetitive elements or other markers. Microsatellites typically include a 1-6 nucleotide core element within SEQ ID NO:1 through SEQ ID NO:304905 that are tandemly repeated from one to many thousands of times. A different “allele” occurs at an SSR locus as a result of changes in the number of times a core element is repeated, altering the length of the repeat region, (Brown et al., Methods of Genome Analysis in Plants, (ed.) Jauhar, CRC Press, Inc, Boca Raton, Fla., USA; London, England, UK, pp. 147-159, (1996), the entirety of which is herein incorporated by reference). SSR loci occur throughout plant genomes, and specific repeat motifs occur at different levels of abundance than those found in animals. The relative frequencies of all SSRs with repeat units of 1-6 nucleotides have been surveyed. The most abundant SSR is AAAAAT followed by Aₙ, AGₙ AAT, AAC, AGC, AAG, AATT, AAAT and AC. On average, 1 SSR is found every 21 and 65 kb in dicots and monocots. Fewer CG nucleotides are found in dicots than in monocots. There is no correlation between abundance of SSRs and nuclear DNA content. The abundance of all tri and tetranucleotide SSR combination jointly have been reported to be equivalent to that of the total di-nucleotide combinations. Mono- di- and tetra-nucleotide repeats are all located in noncoding regions of DNA while 57% of those trinucleotide SSRs containing CG were located within gene coding regions. All repeated trinucleotide SSRs composed entirely of AT are found in noncoding regions, (Brown et al., Methods of Genome Analysis in Plants, ed. Jauhar, CRC Press, Inc, Boca Raton, Fla., USA; London, England, UK, pp. 147-159, (1996).

Microsatellites can be observed in SEQ NO:1 to SEQ NO: 304905 or complements thereof by using the BLASTN program to examine sequences for the presence/absence of microsatellites. In this system, raw sequence data is searched through databases, which store SSR markers collected from publications and 692 classes of di-, tri and tetranucleotide repeat markers generated by computer. Microsatellites can also be observed by screening the BAC library of the present invention by colony or plaque hybridization with a labeled probe containing microsatellite markers; isolating positive clones and sequencing the inserts of the positive clones; suitable primers flanking the microsatellite markers.

Single nucleotide polymorphisms (SNPs) are single base changes in genomic DNA sequence. They generally occur at greater frequency than other markers and are spaced with a greater uniformity throughout a genome than other reported forms of polymorphism. The greater frequency and uniformity of SNPs means that there is greater probability that such a polymorphism will be found near or in a genetic locus of interest than would be the case for other polymorphisms. SNPs are located in protein-coding regions and noncoding regions of a genome. Some of these SNPs may result in defective or variant protein expression (e.g., as a result of mutations or defective splicing). Analysis (genotyping) of characterized SNPs can require only a plus/minus assay rather than a lengthy measurement, permitting easier automation.

SNPs can be characterized using any of a variety of methods. Such methods include the direct or indirect sequencing of the site, the use of restriction enzymes (Botstein et al., Am. J. Hum. Genet. 32:314-331 (1980), the entirety of which is herein incorporated reference; Konieczny and Ausubel, Plant J. 4:403-410 (1993), the entirety of which is herein incorporated by reference), enzymatic and chemical mismatch assays (Myers et al., Nature 313:495-498 (1985), the entirety of which is herein incorporated by reference), allele-specific PCR (Newton et al., Nucl. Acids Res. 17:2503-2516 (1989), the entirety of which is herein incorporated by reference; Wu et al., Proc. Natl. Acad. Sci. USA 86:2757-2760 (1989), the entirety of which is herein incorporated by reference), ligase chain reaction (Barany, Proc. Natl. Acad. Sci. USA 88:189-193 (1991), the entirety of which is herein incorporated by reference), single-strand conformation polymorphism analysis (Labrune et al., Am. J. Hum. Genet. 48: 1115-1120 (1991), the entirety of which is herein incorporated by reference), primer-directed nucleotide incorporation assays (Kuppuswami et al., Proc. Natl. Acad. Sci. USA 88:1143-1147 (1991), the entirety of which is herein incorporated by reference), dideoxy fingerprinting (Sarkar et al., Genomics 13:441-443 (1992), the entirety of which is herein incorporated by reference), solid-phase ELISA-based oligonucleotide ligation assays (Nikiforov et al., Nucl. Acids Res. 22:4167-4175 (1994), the entirety of which is herein incorporated by reference), oligonucleotide fluorescence-quenching assays (Livak et al., PCR Methods Appl. 4:357-362 (1995a), the entirety of which is herein incorporated by reference), 5′-nuclease allele-specific hybridization TaqMan™ assay (Livak et al., Nature Genet. 9:341-342 (1995), the entirety of which is herein incorporated by reference), template-directed dye-terminator incorporation (TDI) assay (Chen and Kwok, Nucl. Acids Res. 25:347-353 (1997), the entirety of which is herein incorporated by reference), allele-specific molecular beacon assay (Tyagi et al., Nature Biotech. 16: 49-53 (1998), the entirety of which is herein incorporated by reference), PinPoint assay (Haff and Smirnov, Genome Res. 7: 378-388 (1997), the entirety of which is herein incorporated by reference), and dCAPS analysis (Neff et al., Plant J. 14:387-392 (1998), the entirety of which is herein incorporated by reference).

SNPs can be observed by examining sequences of overlapping clones in the BAC library according to the method described by Taillon-Miller et al. Genome Res. 8:748-754 (1998), the entirety of which is herein incorporated). SNPs can also be observed by screening the BAC library of the present invention by colony or plaque hybridization with a labeled probe containing SNP markers; isolating positive clones and sequencing the inserts of the positive clones; suitable primers flanking the SNP markers.

Genetic markers of the present invention include “dominant” or “codominant” markers. “Codominant markers” reveal the presence of two or more alleles (two per diploid individual) at a locus. “Dominant markers” reveal the presence of only a single allele per locus. The presence of the dominant marker phenotype (e.g., a band of DNA) is an indication that one allele is present in either the homozygous or heterozygous condition. The absence of the dominant marker phenotype (e.g., absence of a DNA band) is merely evidence that “some other” undefined allele is present. In the case of populations where individuals are predominantly homozygous and loci are predominately dimorphic, dominant and codominant markers can be equally valuable. As populations become more heterozygous and multi-allelic, codominant markers often become more informative of the genotype than dominant markers.

In addition to SSRs and SNPs, repetitive elements can be used as markers. For most eukaryotes, interspersed repeat sequence elements are typically mobile genetic elements (Wright et al., Genetics 142:569-578 (1996), the entirety of which is herein incorporated by reference). They are ubiquitous in most living organisms and are present in copy numbers ranging from just a few elements to tens or hundreds or thousands per genome. In the latter case, they can represent a major fraction of the genome. For example, transposable elements have been estimated to make up greater than 50% of the maize genome (Kidwell, and Lisch Proc. Natl. Acad. Sci. (U.S.A.) 94:7704-7711 (1997), the entirety of which is herein incorporated by reference).

Transposable elements are classified in families according to their sequence similarity. Two major classes are distinguished by their differing modes of transposition. Class I elements are retroelements that use reverse transcriptase to transpose by means of an RNA intermediate. They include long terminal repeat retrotransposons and long and short interspersed elements (LINES and SINES, respectively). Class II elements transpose directly from DNA to DNA and include transposons such as the Activator-Dissociation (Ac-Ds) family in maize, the P element in Drosophila and the Tc-1 element in Caenhorabditis elegans. Additionally, a category of transposable elements has been discovered whose transposition mechanism is not yet known. These miniature inverted-repeat transposable elements (MITEs) have some properties of both class I and II elements. They are short (100-400 bp in length) and none so far has been found to have any coding potential. They are present in high copy number (3,000-10,000) per genome and have target site preferences for TAA or TA in plants (Kidwell and Lisch, Proc. Natl. Acad. Sci. (U.S.A.) 94:7704-7711 (1997)).

Insertion elements are found in two areas of the genome. Some are located in regions distant from gene sequences such as in the heterochromatin or in regions between genes; other repeat elements are found in or near single copy sequences. The insertion of an Ac-Ds element into wx-m9, an allele of the waxy locus in maize is an example of a repetitive element found within a coding region. The effect of this insertion is attenuated by the loss through splicing of the transposable element after transcription (Kidwell and Lisch, Proc. Natl. Acad. Sci. (U.S.A.) 94:7704-7711 (1997)). The BAC nucleic acid molecules of the present invention include BAC nucleic acid molecules having inserts with two defined ends (STCs) containing complex repeat elements as set forth in Table B of U.S. application Ser. No. 09/521,640, which is herein incorporated by reference in its entirety.

The genetic variability resulting from transposable elements ranges from changes in the size and arrangement of whole genomes to changes in single nucleotides. They may produce major effects on phenotypic traits or small silent changes detectable only at the DNA sequence level. Transposable elements may also produce variation when they excise, leaving small footprints of their previous presence (Kidwell and Lisch, Proc. Natl. Acad. Sci. (U.S.A.) 94:7704-7711 (1997)).

In addition, other markers such as AFLP markers, RFLP markers, RAPD markers, phenotypic markers or isozyme markers can be utilized (Walton, Seed World 22-29 (July, 1993), the entirety of which is herein incorporated by reference; Burow and Blake, Molecular Dissection of Complex Traits, 13-29, Eds. Paterson, CRC Press, New York (1988), the entirety of which is herein incorporated by reference). DNA markers can be developed from nucleic acid molecules using restriction endonucleases, the PCR and/or DNA sequence information. RFLP markers result from single base changes or insertions/deletions. These codominant markers are highly abundant in plant genomes, have a medium level of polymorphism and are developed by a combination of restriction endonuclease digestion and Southern blotting hybridization. CAPS are similarly developed from restriction nuclease digestion but only of specific PCR products. These markers are also codominant, have a medium level of polymorphism and are highly abundant in the genome. The CAPS result from single base changes and insertions/deletions. Another marker type, RAPDs, are developed from DNA amplification with random primers and result from single base changes and insertions/deletions in plant genomes. They are dominant markers with a medium level of polymorphisms and are highly abundant. AFLP markers require using the PCR on a subset of restriction fragments from extended adapter primers. These markers are both dominant and codominant, are highly abundant in genomes and exhibit a medium level of polymorphism. SSRs require DNA sequence information. These codominant markers result from repeat length changes, are highly polymorphic, and do not exhibit as high a degree of abundance in the genome as CAPS, AFLPs and RAPDs. SNPs also require DNA sequence information. These codominant markers result from single base substitutions. They are highly abundant and exhibit a medium of polymorphism (Rafalski, et al., In: Nonmammalian Genomic Analysis, ed. Birren and Lai, Academic Press, San Diego, Calif., pp. 75-134 (1996), the entirety of which is herein incorporated by reference). Methods to isolate such markers are known in the art.

Long Terminal repeat retrotransposons and MITEs have been found to be associated with the genes of many plants where some of the transposable elements contribute regulatory sequences. MITEs such as the Tourist element in maize and the Stowaway element in Sorghum are found frequently in the 5′ and 3′ noncoding regions of genes and are frequently associated with the regulatory regions of genes of diverse flowering plants (Kidwell and Lisch, Proc. Natl. Acad. Sci. (U.S.A.) 94:7704-7711 (1997)). It is understood that one or more of the Long Terminal repeat retrotransposons and/or MITES may be a marker, and even more preferably a marker for a gene.

(a)(2) Nucleic Acid Molecules Comprising Regulatory Elements

Another class of agents of the present invention are nucleic acid molecules having promoter regions or partial promoter regions within SEQ ID NO: 1 through SEQ ID NO: 304905. Such promoter regions are typically found upstream of the trinucleotide ATG sequence at the start site of a protein coding region.

As used herein, a promoter region is a region of a nucleic acid molecule that is capable, when located in cis to a nucleic acid sequence that encodes for a protein or fragment thereof to finction in a way that directs expression of one or more mRNA molecules that encodes for the protein or fragment thereof.

Promoters of the present invention can include between about 300 bp upstream and about 10 kb upstream of the trinucleotide ATG sequence at the start site of a protein coding region. Promoters of the present invention can preferably include between about 300 bp upstream and about 5 kb upstream of the trinucleotide ATG sequence at the start site of a protein coding region. Promoters of the present invention can more preferably include between about 300 bp upstream and about 2 kb upstream of the trinucleotide ATG sequence at the start site of a protein coding region. Promoters of the present invention can include between about 300 bp upstream and about 1 kb upstream of the trinucleotide ATG sequence at the start site of a protein coding region. While in many circumstances a 300 bp promoter may be sufficient for expression, additional sequences may act to further regulate expression, for example, in response to biochemical, developmental or environmental signals.

It is also preferred that the promoters of the present invention contain a CAAT and a TATA cis element. Moreover, the promoters of the present invention can contain one or more cis elements in addition to a CAAT and a TATA box.

By “regulatory element” it is intended a series of nucleotides that determines if, when, and at what level a particular gene is expressed. The regulatory DNA sequences specifically interact with regulatory or other proteins. Many regulatory elements act in cis (“cis elements”) and are believed to affect DNA topology, producing local conformations that selectively allow or restrict access of RNA polymerase to the DNA template or that facilitate selective opening of the double helix at the site of transcriptional initiation. Cis elements occur within, but are not limited to promoters, and promoter modulating sequences (inducible elements). Cis elements can be identified using known cis elements as a target sequence or target motif in the BLAST programs of the present invention.

Promoters of the present invention include homologues of cis elements known to effect gene regulation that show homology with the nucleic acid molecules of the present invention. These cis elements include, but are not limited to, oxygen responsive cis elements (Cowen, et al., J. Biol. Chem. 268(36):26904-26910 (1993) the entirety of which is herein incorporated by reference), light regulatory elements (Bruce and Quaill, Plant Cell 2 (11):1081-1089 (1990) the entirety of which is herein incorporated by reference; Bruce, et al., EMBO J. 10:3015-3024 (1991), the entirety of which is herein incorporated by reference; Rocholl, et al., Plant Sci. 97:189-198 (1994), the entirety of which is herein incorporated by reference; Block, et al., Proc. Natl. Acad. Sci. (U.S.A.) 87:5387-5391 (1990), the entirety of which is herein incorporated by reference; Giuliano, et al., Proc. Natl. Acad. Sci. (U.S.A.) 85:7089-7093 (1988), the entirety of which is herein incorporated by reference; Staiger, et al., Proc. Natl. Acad. Sci. (U.S.A.) 86:6930-6934 (1989), the entirety of which is herein incorporated by reference; Izawa, et al., Plant Cell 6:1277-1287 (1994), the entirety of which is herein incorporated by reference; Menkens, et al., Trends in Biochemistry 20:506-510 (1995), the entirety of which is herein incorporated by reference; Foster, et al., FASEB J. 8:192-200 (1994), the entirety of which is herein incorporated by reference; Plesse, et al., Mol Gen Gene 254:258-266 (1997), the entirety of which is herein incorporated by reference; Green, et al., EMBO J. 6:2543-2549 (1987), the entirety of which is herein incorporated by reference; Kuhlemeier et al., Ann. Rev Plant Physiol. 38:221-257 (1987), the entirety of which is herein incorporated by reference; Villain et al., J. Biol. Chem. 271:32593-32598 (1996), the entirety of which is herein incorporated by reference; Lam et al., Plant Cell 2:857-866 (1990), the entirety of which is herein incorporated by reference; Gilmartin, et al., Plant Cell 2:369-378 (1990), the entirety of which is herein incorporated by reference; Datta, et al., Plant Cell 1:1069-1077 (1989) the entirety of which is herein incorporated by reference; Gilmartin, et al., Plant Cell 2:369-378 (1990), the entirety of which is herein incorporated by reference; Castresana, et al., EMBO J. 7:1929-1936 (1988), the entirety of which is herein incorporated by reference; Ueda, et al., Plant Cell 1:217-227 (1989), the entirety of which is herein incorporated by reference; Terzaghi, et al., Annu. Rev. Plant Physiol. Plant Mol. Biol. 46:445-474 (1995), the entirety of which is herein incorporated by reference; Green et al., EMBO J. 6:2543-2549 (1987), the entirety of which is herein incorporated by reference; Villain, et al., J. Biol. Chem. 271:32593-32598 (1996), the entirety of which is herein incorporated by reference; Tjaden, et al., Plant Cell 6:107-118 (1994), the entirety of which is herein incorporated by reference; Tjaden, et al., Plant Physiol. 108:1109-1117 (1995), the entirety of which is herein incorporated by reference; Ngai, et al., Plant J. 12:1021-1234 (1997), the entirety of which is herein incorporated by reference; Bruce, et al., EMBO J. 10:3015-3024 (1991), the entirety of which is herein incorporated by reference; Ngai, et al., Plant J. 12:1021-1034 (1997), the entirety of which is herein incorporated by reference), elements responsive to gibberellin, (Muller, et al., J. Plant Physiol. 145:606-613 (1995), the entirety of which is herein incorporated by reference; Croissant, et al., Plant Science 116:27-35 (1996), the entirety of which is herein incorporated by reference; Lohmer, et al., EMBO J. 10:617-624 (1991), the entirety of which is herein incorporated by reference; Rogers, et al., Plant Cell 4:1443-1451 (1992), the entirety of which is herein incorporated by reference; Lanahan et al., Plant Cell 4:203-211 (1992) the entirety of which is herein incorporated by reference; Skriver et al., Proc. Natl. Acad. Sci. (U.S.A.) 88:7266-7270 (1991) the entirety of which is herein incorporated by reference; Gilmartin, et al., Plant Cell 2:369-378 (1990), the entirety of which is herein incorporated by reference; Huang, et al., Plant Mol. Biol. 14:655-668 (1990), the entirety of which is herein incorporated by reference, Gubler, et al., Plant Cell 7:1879-1891 (1995), the entirety of which is herein incorporated by reference), elements responsive to abscisic acid, (Busk, et al., Plant Cell 9:2261-2270 (1997), the entirety of which is herein incorporated by reference; Guiltinan, et al., Science 250:267-270 (1990), the entirety of which is herein incorporated by reference; Shen, et al., Plant Cell 7:295-307 (1995) the entirety of which is herein incorporated by reference; Shen et al., Plant Cell 8:1107-1119 (1996), the entirety of which is herein incorporated by reference; Seo et al., Plant Mol. Biol. 27:1119-1131 (1995), the entirety of which is herein incorporated by reference; Marcotte et al., Plant Cell 1:969-976 (1989) the entirety of which is herein incorporated by reference; Shen et al., Plant Cell 7:295-307 (1995), the entirety of which is herein incorporated by reference; Iwasaki et al., Mol Gen Genet 247:391-398 (1995), the entirety of which is herein incorporated by reference; Hattori et al., Genes Dev. 6:609-618 (1992), the entirety of which is herein incorporated by reference; Thomas et al., Plant Cell 5:1401-1410 (1993), the entirety of which is herein incorporated by reference), elements similar to abscisic acid responsive elements, (Ellerstrom et al., Plant Mol. Biol. 32:1019-1027 (1996), the entirety of which is herein incorporated by reference), auxin responsive elements (Liu et al., Plant Cell 6:645-657 (1994) the entirety of which is herein incorporated by reference; Liu et al., Plant Physiol. 115:397-407 (1997), the entirety of which is herein incorporated by reference; Kosugi et al., Plant J. 7:877-886 (1995), the entirety of which is herein incorporated by reference; Kosugi et al., Plant Cell 9:1607-1619 (1997), the entirety of which is herein incorporated by reference; Ballas et al., J. Mol. Biol. 233:580-596 (1993), the entirety of which is herein incorporated by reference), a cis element responsive to methyl jasmonate treatment (Beaudoin and Rothstein, Plant Mol. Biol. 33:835-846 (1997), the entirety of which is herein incorporated by reference), a cis element responsive to abscisic acid and stress response (Straub et al., Plant Mol. Biol. 26:617-630 (1994), the entirety of which is herein incorporated by reference), ethylene responsive cis elements (Itzhaki et al., Proc. Natl. Acad. Sci. (U.S.A.) 91:8925-8929 (1994), the entirety of which is herein incorporated by reference; Montgomery et al., Proc. Acad. Sci. (U.S.A.) 90:5939-5943 (1993), the entirety of which is herein incorporated by reference; Sessa et al., Plant Mol. Biol. 28:145-153 (1995), the entirety of which is herein incorporated by reference; Shinshi et al., Plant Mol. Biol. 27:923-932 (1995), the entirety of which is herein incorporated by reference), salicylic acid cis responsive elements, (Strange et al., Plant J. 11:1315-1324 (1997), the entirety of which is herein incorporated by reference; Qin et al., Plant Cell 6:863-874 (1994), the entirety of which is herein incorporated by reference), a cis element that responds to water stress and abscisic acid (Lam et al., J. Biol. Chem. 266:17131-17135 (1991), the entirety of which is herein incorporated by reference; Thomas et al., Plant Cell 5:1401-1410 (1993), the entirety of which is herein incorporated by reference; Pla et al., Plant Mol Biol 21:259-266 (1993), the entirety of which is herein incorporated by reference), a cis element essential for M phase-specific expression (Ito et al., Plant Cell 10:331-341 (1998), the entirety of which is herein incorporated by reference), sucrose responsive elements (Huang et al., Plant Mol. Biol. 14:655-668 (1990), the entirety of which is herein incorporated by reference; Hwang et al., Plant Mol Biol 36:331-341 (1998), the entirety of which is herein incorporated by reference; Grierson et al., Plant J. 5:815-826 (1994), the entirety of which is herein incorporated by reference), heat shock response elements (Pelham et al., Trends Genet. 1:31-35 (1985), the entirety of which is herein incorporated by reference), elements responsive to auxin and/or salicylic acid and also reported for light regulation (Lam et al., Proc. Natl. Acad. Sci. (U.S.A.) 86:7890-7897 (1989), the entirety of which is herein incorporated by reference; Benfey et al., Science 250:959-966 (1990), the entirety of which is herein incorporated by reference), elements responsive to ethylene and salicylic acid (Ohme-Takagi et al., Plant Mol. Biol. 15:941-946 (1990), the entirety of which is herein incorporated by reference), elements responsive to wounding and abiotic stress (Loake et al., Proc. Natl. Acad. Sci. (U.S.A.) 89:9230-9234 (1992), the entirety of which is herein incorporated by reference; Mhiri et al., Plant Mol. Biol. 33:257-266 (1997), the entirety of which is herein incorporated by reference), antioxidant response elements (Rushmore et al., J. Biol. Chem. 266:11632-11639, the entirety of which is herein incorporated by reference; Dalton et al., Nucleic Acids Res. 22:5016-5023 (1994), the entirety of which is herein incorporated by reference), Sph elements (Suzuki et al., Plant Cell 9:799-807 1997), the entirety of which is herein incorporated reference), Elicitor responsive elements, (Fukuda et al., Plant Mol. Biol. 34:81-87 (1997), the entirety of which is herein incorporated by reference; Rushton et al., EMBO J. 15:5690-5700 (1996), the entirety of which is herein incorporated by reference), metal responsive elements (Stuart et al., Nature 317:828-831 (1985), the entirety of which is herein incorporated by reference; Westin et al., EMBO J. 7:3763-3770 (1988), the entirety of which is herein incorporated by reference; Thiele et al., Nucleic Acids Res. 20:1183-1191 (1992), the entirety of which is herein incorporated by reference; Faisst et al., Nucleic Acids Res. 20:3-26 (1992), the entirety of which is herein incorporated by reference), low temperature responsive elements, (Baker et al., Plant Mol. Biol. 24:701-713 (1994), the entirety of which is herein incorporated by reference; Jiang et al., Plant Mol. Biol. 30:679-684 (1996), the entirety of which is herein incorporated by reference; Nordin et al., Plant Mol. Biol. 21:641-653 (1993), the entirety of which is herein incorporated by reference; Zhou etal., J. Biol. Chem. 267:23515-23519 (1992), the entirety of which is herein incorporated by reference), drought responsive elements, (Yamaguchi et al., Plant Cell 6:251-264 (1994), the entirety of which is herein incorporated by reference; Wang et al., Plant Mol. Biol. 28:605-617 (1995), the entirety of which is herein incorporated by reference; Bray E A, Trends in Plant Science 2:48-54 (1997), the entirety of which is herein incorporated by reference) enhancer elements for glutenin, (Colot et al., EMBO J. 6:3559-3564 (1987), the entirety of which is herein incorporated by reference; Thomas et al., Plant Cell 2:1171-1180 (1990), the entirety of which is incorporated by reference; Kreis et al., Philos. Trans. R. Soc. Lond., B314:355-365 (1986), the entirety of which is herein incorporated by reference), light-independent regulatory elements, (Lagrange et al., Plant Cell 9:1469-1479 (1997), the entirety of which is herein incorporated by reference; Villain et al., J. Biol. Chem. 271:32593-32598 (1996), the entirety of which is herein incorporated by reference), OCS enhancer elements, (Bouchez et al., EMBO J. 8:4197-4204 (1989), the entirety of which is herein incorporated by reference; Foley et al., Plant J. 3:669-679 (1993), the entirety of which is herein incorporated by reference), ACGT elements, (Foster et al., FASEB J. 8:192-200 (1994), the entirety of which is herein incorporated by reference; Izawa et al., Plant Cell 6:1277-1287 (1994), the entirety of which is herein incorporated by reference; Izawa et al., J. Mol. Biol. 230:1131-1144 (1993) the entirety of which is herein incorporated by reference), negative cis elements in plastid related genes, (Zhou et al., J. Biol. Chem. 267:23515-23519 (1992), the entirety of which is herein incorporated by reference; Lagrange et al., Mol. Cell Biol. 13:2614-2622 (1993), the entirety of which is herein incorporated by reference; Lagrange et al., Plant Cell 9:1469-1479 (1997), the entirety of which is herein incorporated by reference; Zhou et al., J. Biol. Chem. 267:23515-23519 (1992), the entirety of which is herein incorporated by reference), prolamin box elements, (Forde et al., Nucleic Acids Res. 13:7327-7339 (1985), the entirety of which is herein incorporated by reference; Colot et al., EMBO J. 6:3559-3564 (1987), the entirety of which is herein incorporated by reference; Thomas et al., Plant Cell 2:1171-1180 (1990), the entirety of which is herein incorporated by reference; Thompson et al., Plant Mol. Biol. 15:755-764 (1990), the entirety of which is herein incorporated by reference; Vicente et al., Proc. Natl. Acad. Sci. (U.S.A.) 94:7685-7690 (1997), the entirety of which is herein incorporated by reference), elements in enhancers from the IgM heavy chain gene (Gillies et al., Cell 33:717-728 (1983), the entirety of which is herein incorporated by reference; Whittier et al., Nucleic Acids Res. 15:2515-2535 (1987), the entirety of which is herein incorporated by reference.

(a)(3) Nucleic Acid Molecules Comprising Genes or Fragments Thereof

Nucleic acid molecules of the present invention can comprise one or more genes or fragments thereof. Such genes or fragments thereof include homologues of known genes or protein coding regions in other organisms or genes or fragments thereof that elicit only limited or no matches with known genes or protein coding regions.

Genomic sequences can be screened for the presence of protein homologues or genes utilizing one or a number of different search algorithms have that been developed, one example of which are the suite of programs referred to as BLAST programs. Other examples of suitable programs that can be utilized are known in the art, several of which are described above in the Background and under the section titled “Uses of the Agents of the Invention.” In addition, unidentified reading frames may be screened for protein coding regions by prediction software such as GenScan, which is located at the website gnomic.standford.edu/GENSCANW.html.

In a preferred embodiment of the present invention, the Glycine max protein or fragment thereof of the present invention is a homologue of another plant protein. In another preferred embodiment of the present invention, the Glycine max protein or fragment thereof of the present invention is a homologue of a fungal protein. In another preferred embodiment of the present invention, the Glycine max protein or fragment thereof of the present invention is a homologue of a mammalian protein. In another preferred embodiment of the present invention, the Glycine max protein or fragment thereof of the present invention is a homologue of a bacterial protein.

In a preferred embodiment of the present invention, the Glycine max protein or fragments thereof or nucleic acid molecule or fragment thereof has a BLAST score of more than 200, preferably a BLAST score of more than 300, even more preferably a BLAST score of more than 400.

In another preferred embodiment of the present invention, the nucleic acid molecule encoding the Glycine max protein or fragment thereof and/or nucleic acid molecule or fragment thereof exhibits a % identity with its homologue of between about 25% and about 40%, more preferably of between about 40 and about 70%, even more preferably of between about 70% and about 90%, and even more preferably between about 90% and 99%. In another preferred embodiment, of the present invention, the Glycine max the nucleic acid molecule encoding the Glycine max protein or fragment thereof exhibits a % identity with its homologue of 100%.

In a preferred embodiment of the present invention, the Glycine max protein or fragment thereof or nucleic acid molecule or fragment thereof exhibits a % coverage of between about 0% and about 33%, more preferably of between about 34% and about 66%, and even more preferably of between about 67% and about 100%.

Genomic sequences can be screened for the presence of proteins utilizing one or a number of different search algorithms have that been developed, one example of which are the suite of programs referred to as BLAST programs. Other examples of suitable programs that can be utilized are known in the art, several of which are described above in the Background. Nucleic acid molecules of the present invention also include non-Glycine max homologues. Preferred non-Glycine max homologues are selected from the group consisting of alfalfa, Arabidopsis barley, Brassica, broccoli, cabbage, citrus, cotton, garlic, oat, oilseed rape, onion, canola, flax, an ornamental plant, maize, pea, peanut, pepper, potato, rice, rye, sorghum, strawberry, sugarcane, sugarbeet, tomato, wheat, poplar, pine, fir, eucalyptus, apple, lettuce, lentils, grape, banana, tea, turf grasses, sunflower, oil palm, and Phaseolus.

In a preferred embodiment, nucleic acid molecules having SEQ ID NO: 1 through SEQ ID NO: 304905 or complements and fragments of either can be utilized to obtain such homologues.

The degeneracy of the genetic code allows different nucleic acid sequences to code for the same protein or peptide, e.g. see U.S. Pat. No. 4,757,006, the entirety of which is herein incorporated by reference. As used herein a nucleic acid molecule is degenerate of another nucleic acid molecule when the nucleic acid molecules encode for the same amino acid sequences but comprise different nucleotide sequences. An aspect of the present invention is that the nucleic acid molecules of the present invention include nucleic acid molecules that are degenerate from the STCs of this invention.

A further aspect of the present invention comprises one or more nucleic acid molecules which differ in nucleic acid sequence from those of a STC of this invention due to the degeneracy in the genetic code in that they encode the same protein but differ in nucleic acid sequence or a protein having one or more conservative amino acid residue. Codons capable of coding for such conservative substitutions are known in the art. For instance, serine is a conservative substitute of alanine and threonine is a conservative substitute for serine.

(a)(4) Nucleic Acid Molecules Comprising Introns and/or Intron/Exon Junctions

Nucleic acid molecules of the present invention can comprise an intron and/or one or more intron/exon junction. Sequences of the present invention can be screened for introns and intron/exon junctions utilizing one or a number of different search algorithms that have that been developed, one example of which are the suite of programs referred to as BLAST programs. Other examples of suitable programs that can be utilized are known in the art, several of which are described above in the Background and in the section entitled “Uses of the Agents of the Present Invention”.

(a)(4) Protein and Peptide Molecules

A class of agents comprises one or more of the protein or peptide molecules encoded by SEQ ID NO: 1 through SEQ ID NO: 304905, fragments thereof or complements thereof or one or more of the proteins encoded by a nucleic acid molecule or fragment thereof or peptide molecules encoded by other nucleic acid agents of the present invention. Protein and peptide molecules can be identified using known protein or peptide molecules as a target sequence or target motif in the BLAST programs of the present invention. In a preferred embodiment, the protein or peptide molecules of the present invention are derived from Glycine max (soybean) and more preferably Glycine max, genotype A3244.

As used herein, the term “protein molecule” or “peptide molecule” includes any molecule that comprises five or more amino acids. It is well known in the art that proteins or peptides may undergo modification, including post-translational modifications, such as, but not limited to, disulfide bond formation, glycosylation, phosphorylation, or oligomerization. Thus, as used herein, the term “protein molecule” or “peptide molecule” includes any protein molecule that is modified by any biological or non-biological process. The terms “amino acid” and “amino acids” refer to all naturally occurring L-amino acids. This definition is meant to include norleucine, ornithine, homocysteine, and homoserine.

One or more of the protein or fragments of peptide molecules may be produced via chemical synthesis, or more preferably, by expression in a suitable bacterial or eukaryotic host. Suitable methods for expression are described by Sambrook, et al., Molecular Cloning, A Laboratory Manual, 2nd Edition, Cold Spring Harbor Press, Cold Spring Harbor, N.Y. (1989), or similar texts.

A “protein fragment” is a peptide or polypeptide molecule whose amino acid sequence comprises a subset of the amino acid sequence of that protein. A protein or fragment thereof that comprises one or more additional peptide regions not derived from that protein is a “fusion” protein. Such molecules may be derivatized to contain carbohydrate or other moieties (such as keyhole limpet hemocyanin, etc.). Fusion protein or peptide molecules of the present invention are preferably produced via recombinant means.

Another class of agents comprises protein or peptide molecules encoded by SEQ ID NO: 1 through SEQ ID NO: 304905 or complements thereof or, fragments or fusions thereof in which conservative, non-essential, or not relevant, amino acid residues have been added, replaced, or deleted. An example of such a homologue is the homologue protein of all non-Glycine max plant species, including but not limited to alfalfa, barley, Brassica, broccoli, cabbage, citrus, cotton, garlic, oat, oilseed rape, onion, canola, flax, maize, an ornamental plant, pea, peanut, pepper, potato, rice, rye, sorghum, strawberry, sugarcane, sugarbeet, tomato, wheat, poplar, pine, fir, eucalyptus, apple, lettuce, peas, lentils, grape, banana, tea, turf grasses, etc. Particularly preferred non-Glycine max plants to utilize for the isolation of homologues would include alfalfa, barley, cotton, corn, oat, oilseed rape, rice, corn, canola, ornamentals, sugarcane, sugarbeet, tomato, potato, wheat, and turf grasses. Such a homologue can be obtained by any of a variety of methods. Most preferably, as indicated above, one or more of the disclosed sequences (SEQ ID NO: 1 through SEQ ID NO: 304905 or complements thereof) will be used to define a pair of primers that may be used to isolate the homologue-encoding nucleic acid molecules from any desired species. Such molecules can be expressed to yield homologues by recombinant means.

(a)(5) Antibodies

One aspect of the present invention concerns antibodies, single-chain antigen binding molecules, or other proteins that specifically bind to one or more of the protein or peptide molecules of the present invention and their homologs, fusions or fragments. Such antibodies may be used to quantitatively or qualitatively detect the protein or peptide molecules of the present invention. As used herein, an antibody or peptide is said to “specifically bind” to a protein or peptide molecule of the present invention if such binding is not competitively inhibited by the presence of non-related molecules. In a preferred embodiment the antibodies of the present invention bind to proteins of the present invention, in a more preferred embodiment of the antibodies of the present invention bind to proteins derived from Glycine max.

Nucleic acid molecules that encode all or part of the protein of the present invention can be expressed, via recombinant means, to yield protein or peptides that can in turn be used to elicit antibodies that are capable of binding the expressed protein or peptide. Such antibodies may be used in immunoassays for that protein. Such protein-encoding molecules, or their fragments may be a “fusion” molecule (i.e., a part of a larger nucleic acid molecule) such that, upon expression, a fusion protein is produced. It is understood that any of the nucleic acid molecules of the present invention may be expressed, via recombinant means, to yield proteins or peptides encoded by these nucleic acid molecules.

The antibodies that specifically bind proteins and protein fragments of the present invention may be polyclonal or monoclonal. It is understood that practitioners are familiar with the standard resource materials which describe specific conditions and procedures for the construction, manipulation and isolation of antibodies (see, for example, Harlow and Lane, Antibodies: A Laboratory Manual, Cold Spring Harbor Press, Cold Spring Harbor, N.Y. (1988), the entirety of which is herein incorporated by reference).

It is understood that any of the antibodies of the present invention can be substantially purified and/or be biologically active and/or recombinant.

Uses of the Agents of the Invention

Nucleic acid molecules of the present invention may be employed to obtain other Glycine max nucleic acid molecules. Such molecules can be readily obtained by using the above-described nucleic acid molecules to screen libraries Glycine max libraries.

Nucleic acid molecules and fragments thereof of the present invention may also be employed to obtain nucleic acid molecule homologs of non-Glycine max species including the nucleic acid molecules that encode, in whole or in part, protein homologs of other species or other organisms, sequences of genetic elements such as promoters and transcriptional regulatory elements.

Nucleic acid molecules and fragments thereof of the present invention may be employed for genetic mapping studies using linkage analysis (genetic markers). A genetic linkage map shows the relative locations of specific DNA markers along a chromosome. Maps are used for the identification of genes associated with genetic diseases or phenotypic traits, comparative genomics, and as a guide for physical mapping. Through genetic mapping, a fine scale linkage map can be developed using DNA markers, and, then, a genomic DNA library of large-sized fragments can be screened with molecular markers linked to the desired trait. In a preferred embodiment of the present invention, the genomic library screened with the nucleic acid molecules of the present invention is a genomic library of Glycine max.

Mapping marker locations is based on the observation that two markers located near each other on the same chromosome will tend to be passed together from parent to offspring. During gamete production, DNA strands occasionally break and rejoin in different places on the same chromosome or on the homologous chromosome. The closer the markers are to each other, the more tightly linked and the less likely a recombination event will fall between and separate them. Recombination frequency thus provides an estimate of the distance between two markers.

In segregating populations, target genes have been reported to have been placed within an interval of 5-10 cM with a high degree of certainty (Tanksley et al., Trends in Genetics 11(2):63-68 (1995), the entirety of which is herein incorporated by reference). The markers defining this interval are used to screen a larger segregating population to identify individuals derived from one or more gametes containing a crossover in the given interval. Such individuals are useful in orienting other markers closer to the target gene. Once identified, these individuals can be analyzed in relation to all molecular markers within the region to identify those closest to the target.

Markers of the present invention can be employed to construct linkage maps and to locate genes with qualitative and quantitative effects. The genetic linkage of additional marker molecules can be established by a genetic mapping model such as, without limitation, the flanking marker model reported by Lander and Botstein, Genetics, 121:185-199 (1989), and the interval mapping, based on maximum likelihood methods described by Lander and Botstein, Genetics, 121:185-199 (1989), the entirety of which is herein incorporated by reference and implemented in the software package MAPMAKER/QTL (Lincoln and Lander, Mapping Genes Controlling Quantitative Traits Using MAPMAKER/QTL, Whitehead Institute for Biomedical Research, Massachusetts, (1990)). Additional software includes Qgene, Version 2.23 (1996), Department of Plant Breeding and Biometry, 266 Emerson Hall, Cornell University, Ithaca, N.Y., the manual of which is herein incorporated by reference in its entirety). Use of the Qgene software is a particularly preferred approach.

A maximum likelihood estimate (MLE) for the presence of a marker is calculated, together with an MLE assuming no QTL effect, to avoid false positives. A log₁₀ of an odds ratio (LOD) is then calculated as: LOD=log₁₀ (MLE for the presence of a QTL/MLE given no linked QTL).

The LOD score essentially indicates how much more likely the data are to have arisen assuming the presence of a QTL than in its absence. The LOD threshold value for avoiding a false positive with a given confidence, say 95%, depends on the number of markers and the length of the genome. Graphs indicating LOD thresholds are set forth in Lander and Botstein, Genetics, 121:185-199 (1989), the entirety of which is herein incorporated by reference and further described by Arús and Moreno-González, Plant Breeding, Hayward, Bosemark, Romagosa (eds.) Chapman & Hall, London, pp. 314-331 (1993).

Additional models can be used. Many modifications and alternative approaches to interval mapping have been reported, including the use of non-parametric methods (Kruglyak and Lander, Genetics, 139:1421-1428 (1995), the entirety of which is herein incorporated by reference). Multiple regression methods or models can be also be used, in which the trait is regressed on a large number of markers (Jansen, Biometrics in Plant Breed, van Oijen, Jansen (eds.) Proceedings of the Ninth Meeting of the Eucarpia Section Biometrics in Plant Breeding, The Netherlands, pp. 116-124 (1994); Weber and Wricke, Advances in Plant Breeding, Blackwell, Berlin, 16 (1994). Procedures combining interval mapping with regression analysis, whereby the phenotype is regressed onto a single putative QTL at a given marker interval, and at the same time onto a number of markers that serve as ‘cofactors,’ have been reported by Jansen and Stam, Genetics, 136:1447-1455 (1994) and Zeng, Genetics, 136:1457-1468 (1994). Generally, the use of cofactors reduces the bias and sampling error of the estimated QTL positions (Utz and Melchinger, Biometrics in Plant Breeding, van Oijen, Jansen (eds.) Proceedings of the Ninth Meeting of the Eucarpia Section Biometrics in Plant Breeding, The Netherlands, pp. 195-204 (1994), thereby improving the precision and efficiency of QTL mapping (Zeng, Genetics, 136:1457-1468 (1994). These models can be extended to multi-environment experiments to analysis genotype-environment interactions (Jansen et al., Theo. Appl. Genet. 91:33-37 (1995).

Selection of an appropriate mapping population is important to map construction. The choice of appropriate mapping population depends on the type of marker systems employed (Tanksley et al., J.P. Gustafson and R. Appels (eds.), Plenum Press, New York, pp. 157-173 (1988), the entirety of which is herein incorporated by reference). Consideration must be given to the source of parents (adapted vs. exotic) used in the mapping population. Chromosome pairing and recombination rates can be severely disturbed (suppressed) in wide crosses (adapted x exotic) and generally yield greatly reduced linkage distances. Wide crosses will usually provide segregating populations with a relatively large array of polymorphisms when compared to progeny in a narrow cross (adapted x adapted).

An F₂ population is the first generation of selfing after the hybrid seed is produced. Usually a single F₁ plant is selfed to generate a population segregating for all the genes in Mendelian (1:2:1) fashion. Maximum genetic information is obtained from a completely classified F₂ population using a codominant marker system (Mather, Measurement of Linkage in Heredity: Methuen and Co., (1938), the entirety of which is herein incorporated by reference). In the case of dominant markers, progeny tests (e.g., F₃, BCF₂) are required to identify the heterozygotes, thus making it equivalent to a completely classified F₂ population. However, this procedure is often prohibitive because of the cost and time involved in progeny testing. Progeny testing of F₂ individuals is often used in map construction where phenotypes do not consistently reflect genotype (e.g., disease resistance) or where trait expression is controlled by a QTL. Segregation data from progeny test populations (e.g., F₃ or BCF₂) can be used in map construction. Marker-assisted selection can then be applied to cross progeny based on marker-trait map associations (F₂, F₃), where linkage groups have not been completely disassociated by recombination events (i.e., maximum disequilibrium).

Recombinant inbred lines (RIL) (genetically related lines; usually >F₅, developed from continuously selfing F₂ lines towards homozygosity) can be used as a mapping population. Information obtained from dominant markers can be maximized by using RIL because all loci are homozygous or nearly so. Under conditions of tight linkage (i.e., about <10% recombination), dominant and co-dominant markers evaluated in RIL populations provide more information per individual than either marker type in backcross populations (Reiter, Proc. Natl. Acad. Sci. (U.S.A.) 89:1477-1481 (1992). However, as the distance between markers becomes larger (i.e., loci become more independent), the information in RIL populations decreases dramatically when compared to codominant markers.

Backcross populations (e.g., generated from a cross between a successful variety (recurrent parent) and another variety (donor parent) carrying a trait not present in the former) can be utilized as a mapping population. A series of backcrosses to the recurrent parent can be made to recover most of its desirable traits. Thus a population is created consisting of individuals nearly like the recurrent parent but each individual carries varying amounts or mosaic of genomic regions from the donor parent. Backcross populations can be useful for mapping dominant markers if all loci in the recurrent parent are homozygous and the donor and recurrent parent have contrasting polymorphic marker alleles (Reiter et al., Proc. Natl. Acad. Sci. (U.S.A.) 89:1477-1481 (1992). Information obtained from backcross populations using either codominant or dominant makers is less than that obtained from F₂ populations because one, rather than two, recombinant gametes are sampled per plant. Backcross populations, however, are more informative (at low marker saturation) when compared to RILs as the distance between linked loci increases in RIL populations (i.e., about 0.15% recombination). Increased recombination can be beneficial for resolution of tight linkages, but may be undesirable in the construction of maps with low marker saturation.

Near-isogenic lines (NIL)(created by many backcrosses to produce an array of individuals that are nearly identical in genetic composition except for the trait or genomic region under interrogation) can be used as a mapping population. In mapping with NILs, only a portion of the polymorphic loci are expected to map to a selected region.

Bulk segregant analysis (BSA) is a method developed for the rapid identification of linkage between markers and traits of interest (Michelmore, et al., Proc. Natl. Acad. Sci. (U.S.A.) 88:9828-9832 (1991). In BSA, two bulked DNA samples are drawn from a segregating population originating from a single cross. These bulks contain individuals that are identical for a particular trait (resistant or susceptible to particular disease) or genomic region but arbitrary at unlinked regions (i.e., heterozygous). Regions unlinked to the target region will not differ between the bulked samples of many individuals in BSA.

Applications for markers in plant breeding include: Quantitative Trait Loci (QTL) mapping (Edwards et al, Genetics 116:113-115 (1987), the entirety of which is herein incorporated by reference); Nienhuis et al, Crop Sci. 27:797-803 (1987); Osborn et al, Theor. Appl. Genet. 73:350-356 (1987); Romero-Severson et al, Use of RFLPs In Analysis of Quantitative Trait Loci In Maize, In Helentjaris and Burr (eds.) pp. 97-102 (1989), the entirety of which is herein incorporated by reference; Young et al, Genetics 120:570-585 (1988), the entirety of which is herein incorporated by reference; Martin et al, Science 243:1725-1728 (1989), the entirety of which is herein incorporated by reference): Sarfatti et al., Theor. Appl Genet. 78:22-26 (1989), the entirety of which is herein incorporated by reference; Tanksley, et al., Biotech. 7:257-264 (1989); Barone et al, Mol. Gen. Genet. 224:177-182 (1990), the entirety of which is herein incorporated by reference); Jung et al, Theor, Appl. Genet. 79:663-672 (1990), the entirety of which is herein incorporated by reference; Keim et al, Genetics 126:735-742 (1990), the entirety of which is herein incorporated by reference, Theor. Appl. Genet. 79:465-369 (1990), the entirety of which is herein incorporated by reference; Paterson et al., Genetics 124:735-742 (1990), the entirety of which is herein incorporated by reference; Martin et al, Proc. Natl. Acad. Sci. (U.S.A.) 88:2336-2340 (1991), the entirety of which is herein incorporated by reference; Messeguer et al, Theor. Appl. Genet. 82:529-536 (1991), the entirety of which is herein incorporated by reference; Michelmore et al, Proc Natl. Acad. Sci. (U.S.A.) 88:9828-9832 (1991), the entirety of which is herein incorporated by reference; Ottaviano et al, Theor. Appl. Genet. 81:713-719 (1991), the entirety of which is herein incorporated by reference; Yu et al, Theor. Appl. Genet. 81:471-476 (1991), the entirety of which is herein incorporated by reference; Diers et al, Crop Sci. 32:77-383 (1992), the entirety of which is herein incorporated by reference, Theor. Appl. Genet. 83:608-612 (1992), the entirety of which is herein incorporated by reference, J. Plant Nut. 15:2127-2136 (1992), the entirety of which is herein incorporated by reference; Doebley et al, Proc. Natl. Acad. Sci. (U.S.A.) 87:9888-9892 (1990), the entirety of which is herein incorporated by reference), screening genetic resource strains for useful quantitative trait alleles and introgression of these alleles into commercial varieties (Beckmann and Soller, Theor. Appl. Genet. 67:35-43 (1983), the entirety of which is herein incorporated by reference; Tanksley et al, (1989) the entirety of which is incorporated by reference), or the mapping of mutations (Rafalski, et al., In: Nonmammalian Genomic Analysis, ed. Birren and Lai, Academic Press, San Diego, Calif., pp. 75-134 (1996). Additionally, markers can be used to characterize transformants or germplasm, as a genetic diagnostic test for plant breeding or to identify individuals or varieties (Soller and Beckmann, Theor. Appl. Genet. 67:25-33 (1983), the entirety of which is herein incorporated by reference; Tanksley et al, 1989). Markers also can be used to obtain information about: (1) the number, effect, and chromosomal location of each gene affecting a trait; (2) effects of multiple copies of individual genes (gene dosage); (3) interaction between/among genes controlling a trait (epistasis); (4) whether individual genes affect more than one trait (pleiotropy); and (5) stability of gene function across environments (G x E interactions).

It is understood that one or more of the nucleic acid molecules of the present invention may in one embodiment be used as markers in genetic mapping. In a preferred embodiment, nucleic acid molecules of the present invention may in one embodiment be used as markers with Glycine max.

The nucleic acid molecules of the present invention may be used for physical mapping. Physical mapping, in conjunction with linkage analysis, can enable the isolation of genes. Physical mapping has been reported to identify the markers closest in terms of genetic recombination to a gene target for cloning. Once a DNA marker is linked to a gene of interest, the chromosome walking technique can be used to find the genes via overlapping clones. For chromosome walking, random molecular markers or established molecular linkage maps are used to conduct a search to localize the gene adjacent to one or more markers. A chromosome walk (Bukanov and Berg, Mo. Microbiol, 11:509-523 (1994), the entirety of which is herein incorporated by reference; Birkenbihl and Vielmetter Nucleic Acids Res. 17:5057-5069 (1989), the entirety of which is herein incorporated by reference; Wenzel and Herrmann, Nucleic Acids Res. 16:8323-8336, (1988), the entirety of which is herein incorporated by reference) is then initiated from the closest linked marker. Starting from the selected clones, labeled probes specific for the ends of the insert DNA are synthesized and used as probes in hybridizations against a representative library. Clones hybridizing with one of the probes are picked and serve as templates for the synthesis of new probes; by subsequent analysis, contigs are produced.

The degree of overlap of the hybridizing clones used to produce a contig can be determined by comparative restriction analysis. Comparative restriction analysis can be carried out in different ways all of which exploit the same principle; two clones of a library are very likely to overlap if they contain a limited number of restriction sites for one or more restriction endonucleases located at the same distance from each other. The most frequently used procedures are, fingerprinting (Coulson et al, Proc. Natl. Acad. Sci. (U.S.A.) 83:7821-7821, (1986), the entirety of which is herein incorporated by reference); Knott et al., Nucleic Acids Res. 16:2601-2612 (1988), the entirety of which is herein incorporated by reference; Eiglmeier et al., Mol. Microbiol. 7(2)::197-206 (1993), the entirety of which is herein incorporated by reference, 1993), restriction fragment mapping (Smith and Birnstiel, Nucleic Acids Res. 3:2387-2398 (1976), the entirety of which is herein incorporated by reference, or the “landmarking” technique (Charlebois et al., J. Mol. Biol. 222:509-524 (1991), the entirety of which is herein incorporated by reference To generate a physical map of a genome with BACs using the fingerprinting technique, a BAC library containing a number of clones equivalent to 4×-20× haploid genome can be used (Zhang and Wing., Plant Mol. Bio. 35:115-127 (1997)). For example, BAC DNA can be purified with the conventional alkaline lysis procedure as used for plasmid DNA purification, digested with the restriction enzyme used for construction of the BAC libraries and end-labeled with ³²P-dATP, digested with Sau3AI and fractionated on a denaturing polyacrylamide gel. The gel is dried to chromatography paper and exposed to X-ray film. Fingerprints are scanned and then converted into database records, according to the positions of each band relative to the bands of the closest molecular-weight marker on a gel. The incoming database of fingerprints are first compared against each other to assemble contigs if overlapped, and then compared against all existing databases to place the incoming BACs and BAC contigs in established contigs if overlapped. The physical length of a contig in kb is estimated according to the number of restriction sites of the enzyme used for the first digestion prior to fragment end labeling

Restriction analysis of a certain clone can be carried out, for example, according to a method originally described by Smith and Berstiel, Nucleic Acids Res. 3:2387-2398 (1976), First, the number and size of cloned restriction fragments to be mapped are determined by complete digestion and agarose gel electrophoresis. Then, the clone is linearized at a unique restriction site outside of the cloned DNA. Aliquots of the linearized molecules are digested to different extents with the enzyme selected for mapping. These partially cut samples are separated on agarose gels, blotted, and hybridized to a labeled fragment of vector DNA. This probe is derived entirely from one side or the other of the unique site used to linearize the clone.

The results show a ladder of DNA fragments that have the same unique end. By repeating these analyses in pairs with all the neighboring intermediate DNA fragments, the correct order of restriction fragments as well as the orientation of the cloned insert can be deduced. The order of restriction fragments produced by restriction enzymes other than the cloning enzyme can be determined similarly. Fragment data from different enzymes are then combined by a computer program and compared with the alignments of other clones of the library (Kohara et al., Cell 50:495-508 (1987), the entirety of which is herein incorporated by reference).

The landmarking technique can be carried out without any labeling and relies on agarose gel analysis. Clones are first digested preferably with a 6 bp specific endonuclease A, if possible with the original clone enzyme. Clones are then digested with a second endonuclease B. Endonuclease B is chosen based on its ability to cut rarely in the genome, for example, on average only once in 30 kbp. Of the fragments generated by digestion of one clone with enzyme A, statistically only a small number (between zero and three fragments) will also be cut by enzyme B. The very specific pattern of those fragments which are produced by double digestion are easily recognized. Any of these fragments which have a restriction site for the rarely cutting endonuclease is called a “landmark” Generally one common landmark is sufficient for defining two overlapping clones.

Alternatively to chromosome walking and the associated comparative restriction analyses methods, chromosome landing also has been reported to be used to locate a gene of interest (Tanksley et al., Trends in Genetics 11(2):63-68 (1995), the entirety of which is herein incorporated by reference. For chromosome landing, a DNA marker is isolated at a physical distance from the targeted gene. High resolution linkage analysis is used to identify such a marker that cosegregates with the gene. The marker is isolated at a distance that is less than the average insert size of the genomic library used for clone isolation. The DNA marker is then used to screen the library and isolate (or “land” on) the clone containing the gene without chromosome walking. Genome coverage of a library can also be determined by cross-hybridization of individual large insert clones by screening a BAC library with single copy RFLP markers distributed randomly across the genome by hybridization. To assure accuracy of the physical map, the markers should be single-copy or of single-locus origin, if multiple-copy.

Chromosome landing of large-insert clones using chromosome-specific DNA markers such as STSs microsatellites, RFLPs, or other markers can correlate physical and genetic maps (Zwick et al., Genetics 148:1983-1992 (1998), the entirety of which is herein incorporated by reference in its entirety). These strategies include chromosome landing of BACs containing markers or BAC contigs by BAC-FISH (Fluorescent In Situ Hybridization), a technique that involves tagging the DNA marker with an observable label. BAC clones giving positive hybridization signals are individually analyzed by FISH to metaphase chromosome spreads. The location of the labeled probe can be detected after it binds to its complementary DNA strand in an intact chromosome. The FISH of a BAC selected from a BAC contig will directly place the BAC contig to a specific chromosome region and establish a linkage relationships of the BAC contig to another BAC contig.

Likewise, BACs and STCs of the present invention can be used for contig mapping (Venter, et al., Nature, 381:364-366 (1996), the entirety of which is herein incorporated by reference). A “seed” BAC insert can be sequenced and then STCs and the corresponding BAC of each STC can be placed on the sequenced insert using the BLASTN program. Marker or gene containing STCs can be determined by the BLASTN program and their corresponding BACs can be hybridized to specific chromosomes using BAC-FISH (Zwick et al., Genetics 148:1983-1992 (1998)).

STCs can be used to identify a minimum tiling path of BACs by computational procedures. Any nucleation sequence (the sequence of an entire BAC, for example) can be electronically compared to a database of STCs to identify the next clones to be sequenced to maximally extend a contig. Chosen STCs need to occupy correct positions in the tiling path. Several factors can contribute to errors in the positioning and selection of these clones. An STC that contains all or part of a repetitive element can appear to align at any part of the growing mosaic which contains that element. One method of selecting the appropriate BAC is to mask out all sections of DNA sequence which are known to be repetitive elements. The sequence symbols of these section are replaced with Ns. These sections of DNA are not used to align the STC. STCs which are completely comprised of Ns are discarded. In this way, the unmasked sections of DNA may be aligned against the growing mosaic without misplacing them due to redundant sequence. A program publicly available, PowerBLAST includes a number of options for masking repetitive elements and low complexity subsequences (Zhang and Madden, Genome Res 7:649-56 (1997), the entirety of which is herein incorporated by reference. cDNA and genomic libraries also can be used as probe sources, thus directly combining the ordering of the genomic DNA with the localization of transcribed sequences. By a simultaneous hybridization to the genomic and back to the transcriptional libraries, results are produced on sequence homologies between transcribed sequences.

It is understood that the nucleic acid molecules of the present invention may in one embodiment be used in physical mapping. In a preferred embodiment, nucleic acid molecules of the present invention may in one embodiment be used in the physical mapping of Glycine max.

Nucleic acid molecules of the present invention can be used in comparative mapping (physical and genetic). Comparative mapping within families provides a method to the degree of sequence conservation, gene order, ploidy of species, ancestral relationships and the rates at which individual genomes are evolving. Comparative mapping has been carried out by cross-hybridizing molecular markers across species within a given family. As in genetic mapping, molecular markers are needed but instead of direct hybridization to mapping filters, the markers are used to select large insert clones from a total genomic DNA library of a related species. The selected clones, each a representative of a single marker, can then be used to physically map the region in the target species. The advantage of this method for comparative mapping is that no mapping population or linkage map of the target species is needed and the clones may also be used in other closely related species. By comparing the results obtained by genetic mapping in model plants, with those from other species, similarities of genomic structure among plants species can be established. Cross-hybridization of RFLP markers have been reported and conserved gene order has been established in many studies. Such macroscopic synteny is utilized for the estimation of correspondence of loci among these crops. These loci include not only Mendelian genes but also Quantitative Trait Loci (QTL) (Mohan et al., Molecular Breeding 3:87-103 (1997), the entirety of which is herein incorporated by reference.

It is understood that markers of the present invention may in another embodiment be used in comparative mapping. In a preferred embodiment the markers of present invention may be used in the comparative mapping of Glycine clandestina, Glycine gracilis, Glycine soja, Glycine tomentella, and Glycine tabaina.

The nucleic acid molecules of the present invention can be used to identify polymorphisms. In one embodiment, one or more of the STC nucleic acid molecules or a BAC nucleic acid molecule (or a sub-fragment of either) may be employed as a marker nucleic acid molecule to identify such polymorphism(s). Alternatively, such polymorphisms can be detected through the use of a marker nucleic acid molecule or a marker protein that is genetically linked to (i.e., a polynucleotide that co-segregates with) such polymorphism(s).

In an alternative embodiment, such polymorphisms can be detected through the use of a marker nucleic acid molecule that is physically linked to such polymorphism(s). For this purpose, marker nucleic acid molecules comprising a nucleotide sequence of a polynucleotide located within 1 mb of the polymorphism(s), and more preferably within 100 kb of the polymorphism(s), and most preferably within 10 kb of the polymorphism(s) can be employed.

The genomes of animals and plants naturally undergo spontaneous mutation in the course of their continuing evolution (Gusella, Ann. Rev. Biochem. 55:831-854 (1986)). A “polymorphism” is a variation or difference in the sequence of the gene or its flanking regions that arises in some of the members of a species. The variant sequence and the “original” sequence co-exist in the species' population. In some instances, such co-existence is in stable or quasi-stable equilibrium.

A polymorphism is thus said to be “allelic,” in that, due to the existence of the polymorphism, some members of a species may have the original sequence (i.e., the original “allele”) whereas other members may have the variant sequence (i.e., the variant “allele”). In the simplest case, only one variant sequence may exist, and the polymorphism is thus said to be di-allelic. In other cases, the species' population may contain multiple alleles, and the polymorphism is termed tri-allelic, etc. A single gene may have multiple different unrelated polymorphisms. For example, it may have a di-allelic polymorphism at one site, and a multi-allelic polymorphism at another site.

The variation that defines the polymorphism may range from a single nucleotide variation to the insertion or deletion of extended regions within a gene. In some cases, the DNA sequence variations are in regions of the genome that are characterized by short tandem repeats (STRS) that include tandem di- or tri-nucleotide repeated motifs of nucleotides. Polymorphisms characterized by such tandem repeats are referred to as “variable number tandem repeat” (“VNTR”) polymorphisms. VNTRs have been used in identity analysis (Weber, U.S. Pat. No. 5,075,217; Armour, et al., FEBS Lett. 307:113-115 (1992); Jones, et al., Eur. J. Haematol. 39:144-147 (1987); Horn, et al., PCT Application WO91/14003; Jeffreys, European Patent Application 370,719; Jeffreys, U.S. Pat. No. 5,175,082; Jeffreys et al., Amer. J. Hum. Genet. 39:11-24 (1986); Jeffreys et al., Nature 316:76-79 (1985); Gray, et al., Proc. R. Acad. Soc. Lond. 243:241-253 (1991); Moore, et al., Genomics 10:654-660 (1991); Jeffreys, et al., Anim. Genet. 18:1-15 (1987); Hillel, et al., Anim. Genet. 20:145-155 (1989); Hillel, et al., Genet. 124:783-789 (1990), all of which are herein incorporated by reference in their entirety).

The detection of polymorphic sites in a sample of DNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis or other means.

The most preferred method of achieving such amplification employs the polymerase chain reaction (“PCR”) (Mullis, et al., Cold Spring Harbor Symp. Quant. Biol. 51:263-273 (1986); Erlich, et al., European Patent Appln. 50,424; European Patent Appln. 84,796, European Patent Application 258,017, European Patent Appln. 237,362; Mullis, European Patent Appln. 201,184; Mullis, et al., U.S. Pat. No. 4,683,202; Erlich., U.S. Pat. No. 4,582,788; and Saiki, et al., U.S. Pat. No. 4,683,194, all of which are herein incorporated by reference), using primer pairs that are capable of hybridizing to the proximal sequences that define a polymorphism in its double-stranded form.

In lieu of PCR, alternative methods, such as the “Ligase Chain Reaction” (“LCR”) may be used (Barany, Proc. Natl. Acad. Sci. (U.S.A.) 88:189-193 (1991), the entirety of which is herein incorporated by reference. LCR uses two pairs of oligonucleotide probes to exponentially amplify a specific target. The sequences of each pair of oligonucleotides is selected to permit the pair to hybridize to abutting sequences of the same strand of the target. Such hybridization forms a substrate for a template-dependent ligase. As with PCR, the resulting products thus serve as a template in subsequent cycles and an exponential amplification of the desired sequence is obtained.

LCR can be performed with oligonucleotides having the proximal and distal sequences of the same strand of a polymorphic site. In one embodiment, either oligonucleotide will be designed to include the actual polymorphic site of the polymorphism. In such an embodiment, the reaction conditions are selected such that the oligonucleotides can be ligated together only if the target molecule either contains or lacks the specific nucleotide that is complementary to the polymorphic site present on the oligonucleotide. Alternatively, the oligonucleotides may be selected such that they do not include the polymorphic site (see, Segev, PCT Application WO 90/01069, the entirety of which is herein incorporated by reference).

The “Oligonucleotide Ligation Assay” (“OLA”) may alternatively be employed (Landegren, et al., Science 241:1077-1080 (1988), the entirety of which is herein incorporated by reference). The OLA protocol uses two oligonucleotides which are designed to be capable of hybridizing to abutting sequences of a single strand of a target. OLA, like LCR, is particularly suited for the detection of point mutations. Unlike LCR, however, OLA results in “linear” rather than exponential amplification of the target sequence.

Nickerson, et al. have described a nucleic acid detection assay that combines attributes of PCR and OLA (Nickerson, et al., Proc. Natl. Acad. Sci. (U.S.A.) 87:8923-8927 (1990), the entirety of which is herein incorporated by reference). In this method, PCR is used to achieve the exponential amplification of target DNA, which is then detected using OLA. In addition, to requiring multiple, and separate, processing steps, one problem associated with such combinations is that they inherit all of the problems associated with PCR and OLA.

Schemes based on ligation of two (or more) oligonucleotides in the presence of nucleic acid having the sequence of the resulting “di-oligonucleotide”, thereby amplifying the di-oligonucleotide, are also known (Wu, et al., Genomics 4:560 (1989), the entirety of which is herein incorporated by reference), and may be readily adapted to the purposes of the present invention.

Other known nucleic acid amplification procedures, such as allele-specific oligomers, branched DNA technology, transcription-based amplification systems, or isothermal amplification methods may also be used to amplify and analyze such polymorphisms (Malek, et al., U.S. Pat. No. 5,130,238; Davey, et al., European Patent Application 329,822; Schuster et al., U.S. Pat. No. 5,169,766; Miller, et al., PCT Application WO 89/06700; Kwoh, et al., Proc. Natl. Acad. Sci. (U.S.A.) 86:1173-1177 (1989); Gingeras, et al., PCT Application WO 88/10315; Walker, et al., Proc. Natl. Acad. Sci. (U.S.A.) 89:392-396 (1992), all of which are herein incorporated by reference in their entirety).

The identification of a polymorphism can be determined in a variety of ways. By correlating the presence or absence of it in an plant with the presence or absence of a phenotype, it is possible to predict the phenotype of that plant. If a polymorphism creates or destroys a restriction endonuclease cleavage site, or if it results in the loss or insertion of DNA (e.g., a VNTR polymorphism), it will alter the size or profile of the DNA fragments that are generated by digestion with that restriction endonuclease. As such, individuals that possess a variant sequence can be distinguished from those having the original sequence by restriction fragment analysis. Polymorphisms that can be identified in this manner are termed “restriction fragment length polymorphisms” (“RFLPs”). RFLPs have been widely used in human and plant genetic analyses (Glassberg, UK Patent Application 2135774; Skolnick, et al., Cytogen. Cell Genet. 32:58-67 (1982); Botstein, et al., Ann. J. Hum. Genet. 32:314-331 (1980); Fischer, et al. (PCT Application W090/13668); Uhlen, PCT Application W090/11369).

Polymorphisms can also be identified by Single Strand Conformation Polymorphism (SSCP) analysis. The SSCP technique is a method capable of identifying most sequence variations in a single strand of DNA, typically between 150 and 250 nucleotides in length (Elles, Methods in Molecular Medicine: Molecular Diagnosis of Genetic Diseases, Humana Press (1996), the entirety of which is herein incorporated by reference); Orita et al., Genomics 5:874-879 (1989), the entirety of which is herein incorporated by reference). Under denaturing conditions a single strand of DNA will adopt a conformation that is uniquely dependent on its sequence conformation. This conformation usually will be different, even if only a single base is changed. Most conformations have been reported to alter the physical configuration or size sufficiently to be detectable by electrophoresis. A number of protocols have been described for SSCP including, but not limited to Lee et al., Anal. Biochem. 205:289-293 (1992), the entirety of which is herein incorporated by reference; Suzuki et al., Anal. Biochem. 192:82-84 (1991), the entirety of which is herein incorporated by reference; Lo et al., Nucleic Acids Research 20:1005-1009 (1992), the entirety of which is herein incorporated by reference; Sarkar et al., Genomics 13:441-443 (1992), the entirety of which is herein incorporated by reference). It is understood that one or more of the nucleic acids of the present invention, may be utilized as markers or probes to detect polymorphisms by SSCP analysis.

Polymorphisms may also be found using a DNA fingerprinting technique called amplified fragment length polymorphism (AFLP), which is based on the selective PCR amplification of restriction fragments from a total digest of genomic DNA to profile that DNA. Vos, et al., Nucleic Acids Res. 23:4407-4414 (1995), the entirety of which is herein incorporated by reference. This method allows for the specific co-amplification of high numbers of restriction fragments, which can be visualized by PCR without knowledge of the nucleic acid sequence.

AFLP employs basically three steps. Initially, a sample of genomic DNA is cut with restriction enzymes and oligonucleotide adapters are ligated to the restriction fragments of the DNA. The restriction fragments are then amplified using PCR by using the adapter and restriction sequence as target sites for primer annealing. The selective amplification is achieved by the use of primers that extend into the restriction fragments, amplifying only those fragments in which the primer extensions match the nucleotide flanking the restriction sites. These amplified fragments are then visualized on a denaturing polyacrylamide gel.

AFLP analysis has been performed on Salix (Beismann, et al., Mol. Ecol. 6:989-993 (1997), the entirety of which is herein incorporated by reference); Acinetobacter (Janssen, et al., Int. J. Syst. Bacteriol 47:1179-1187 (1997), the entirety of which is herein incorporated by reference), Aeromonas popoffi (Huys, et al., Int. J. Syst. Bacteriol. 47:1165-1171 (1997), the entirety of which is herein incorporated by reference), rice (McCouch, et al., Plant Mol. Biol. 35:89-99 (1997), the entirety of which is herein incorporated by reference); Nandi, et al., Mol. Gen. Genet. 255:1-8 (1997); Cho, et al., Genome 39:373-378 (1996), herein incorporated by reference), barley (Hordeum vulgare) (Simons, et al., Genomics 44:61-70 (1997), the entirety of which is herein incorporated by reference; Waugh, et al., Mol. Gen. Genet. 255:311-321 (1997), the entirety of which is herein incorporated by reference; Qi, et al., Mol. Gen. Genet. 254:330-336 (1997), the entirety of which is herein incorporated by reference; Becker, et al., Mol. Gen. Genet. 249:65-73 (1995), the entirety of which is herein incorporated by reference), potato (Van der Voort, et al., Mol. Gen. Genet. 255:438-447 (1997), the entirety of which is herein incorporated by reference; Meksem, et al., Mol. Gen. Genet. 249:74-81 (1995), the entirety of which is herein incorporated by reference), Phytophthora infestans (Van der Lee, et al., Fungal Genet. Biol. 21:278-291 (1997), the entirety of which is herein incorporated by reference), Bacillus anthracis (Keim, et al., J. Bacteriol. 179:818-824 (1997)), Astragalus cremnophylax (Travis, et al., Mol. Ecol. 5:735-745 (1996), the entirety of which is herein incorporated by reference), Arabidopsis (Cnops, et al., Mol. Gen. Genet. 253:32-41 (1996), the entirety of which is herein incorporated by reference), Escherichia coli (Lin, et al., Nucleic Acids Res. 24:3649-3650 (1996), the entirety of which is herein incorporated by reference), Aeromonas (Huys, et al., Int. J. Syst. Bacteriol. 46:572-580 (1996), the entirety of which is herein incorporated by reference), nematode (Folkertsma, et al., Mol. Plant Microbe Interact. 9:47-54 (1996), the entirety of which is herein incorporated by reference), tomato (Thomas, et al., Plant J. 8:785-794 (1995), the entirety of which is herein incorporated by reference), and human (Latorra, et al., PCR Methods Appl. 3:351-358 (1994) the entirety of which is herein incorporated by reference). AFLP analysis has also been used for fingerprinting mRNA (Money, et al., Nucleic Acids Res. 24:2616-2617 (1996), the entirety of which is herein incorporated by reference; Bachem, et al., Plant J. 9:745-753 (1996), the entirety of which is herein incorporated by reference). It is understood that one or more of the nucleic acid molecules of the present invention, may be utilized as markers or probes to detect polymorphisms by AFLP analysis for fingerprinting mRNA.

Polymorphisms may also be found using random amplified polymorphic DNA (RAPD) (Williams et al., Nucl. Acids Res. 18:6531-6535 (1990), the entirety of which is herein incorporated by reference) and cleavable amplified polymorphic sequences (CAPS) (Lyamichev et al., Science 260:778-783 (1993), the entirety of which is herein incorporated by reference). It is understood that one or more of the nucleic acid molecules of the present invention, may be utilized as markers or probes to detect polymorphisms by RAPD or CAPS analysis.

Nucleic acid molecules of the present invention can be used to monitor expression. A microarray-based method for high-throughput monitoring of plant gene expression may be utilized to measure gene-specific hybridization targets. This ‘chip’-based approach involves using microarrays of nucleic acid molecules as gene-specific hybridization targets to quantitatively measure expression of the corresponding plant genes (Schena et al., Science 270:467-470 (1995), the entirety of which is herein incorporated by reference; Shalon, Ph.D. Thesis. Stanford University (1996), the entirety of which is herein incorporated by reference). Every nucleotide in a large sequence can be queried at the same time. Hybridization can be used to efficiently analyze nucleotide sequences.

Several microarray methods have been described. One method compares the sequences to be analyzed by hybridization to a set of oligonucleotides or cDNA molecules representing all possible subsequences (Bains and Smith, J. Theor. Biol. 135:303 (1989), the entirety of which is herein incorporated by reference). A second method hybridizes the sample to an array of oligonucleotide or cDNA probes. An array consisting of oligonucleotides or cDNA molecules complementary to subsequences of a target sequence can be used to determine the identity of a target sequence, measure its amount, and detect differences between the target and a reference sequence. Nucleic acid molecule microarrays may also be screened with protein molecules or fragments thereof to determine nucleic acid molecules that specifically bind protein molecules or fragments thereof.

Additionally, microarrays of BACs may be prepared to sufficiently cover 3× of an entire genome. Such microarrays can be used in a variety of genomics experiments including gene mapping, DNA fingerprinting and promoter identification. Microarrays of genomic DNA can also be used for parallel analysis of genomes at single gene resolution (Lemieux et al., Molecular Breeding 277-289 (1988), the entirety of which is herein incorporated by reference). It is understood that one or more of the molecules of the present invention, preferably one or more of the nucleic acid molecules or protein molecules or fragments thereof of the present invention may be utilized in a genomic microarray based method. In a preferred embodiment of the present invention, one or more of the Glycine max nucleic acid molecules or protein molecules or fragments thereof of the present invention may be utilized in a genomic microarray based method. For example, Genomic Mismatch Scanning (GMS), a hybridization-based method of linkage analysis that allows rapid identification of regions of identity-by-descent between two related individuals, can be carried out with microarrays. GMS is reported to have been used to identify genetically common chromosomal segments based on the ability of these DNA sequences to form extensive regions of mismatch-free heteroduplexes. A series of enzymatic steps, coupled with filter binding, is used to selectively remove heteroduplexes that contain mismatches (i.e., chromosomal regions that do not share identity-by descent.). Fragments of chromosomal DNA representing inherited regions are hybridized to a microarray of ordered genomic clones and positive hybridization signals pinpoint regions of identity-by-descent at high resolution (Lemieux et al., Molecular Breeding 277-289 (1988))

It is understood that one or more of the molecules of the present invention, preferably one or more of the nucleic acid molecules or protein molecules or fragments thereof of the present invention may be utilized in a GMS microarray based method to locate regions of identity-by-descent between related individuals. In a preferred embodiment of the present invention, one or more of the Glycine max nucleic acid molecules or protein molecules or fragments thereof of the present invention may be utilized in a GMS microarray based method to locate regions of identity-by-descent between related individuals. The GMS microarray approach can also be used as a tool to map multigenic traits. For example, in yeast, the entire genomic sequence is known and it has been reported that the genes responsible for growth at elevated temperature, a trait required for the pathogenicity of certain yeast strains, may be determined using GMS (Lemieux et al, Molecular Breeding 277-289 (1988)). By analyzing the inheritance of large numbers of tetrads derived from crosses of pathogenic and wild type strains, all the genes responsible for a yeast strain's ability to grow at 42° C., for example, could be identified.

It is understood that one or more of the molecules of the present invention, preferably one or more of the nucleic acid molecules or protein molecules or fragments thereof of the present invention may be utilized in a GMS microarray based method to map multigenic traits. In a preferred embodiment of the present invention, one or more of the Glycine max nucleic acid molecules or protein molecules or fragments thereof of the present invention may be utilized in a GMS microarray based method to map multigenic traits.

Plant repeat elements may be used with GMS microarraying to identify species specific chromosomes in another species background. For example, the maize genome contains moderately repetitive DNA sequences (ZLRS) representing about 2500 copies per haploid genome; these sequences are present in the genus Zea and absent in other graminaceous species. Ananiev et al., (Proc. Natl. Acad. Sci. (U.S.A.) 94:3526-3529 (1997), all of which are herein incorporated by reference in their entirety) have reported unusual plants with individual maize chromosomes added to a complete oat genome generated by embryo rescue from oat (Avena sativa)×Zea mays crosses. By using highly repetitive maize-specific sequences as probes, Ananiev et al. (1997) were able to selectively isolate cosmid clones containing maize genomic DNA.

It is understood that one or more of the molecules of the present invention, preferably one or more of the nucleic acid molecules or protein molecules or fragments thereof of the present invention may be utilized in a GMS microarray based method using repeat elements to selectively isolate clones containing species specific DNA. In a preferred embodiment of the present invention, one or more of the Glycine max nucleic acid molecules or protein molecules or fragments thereof of the present invention may be utilized in a GMS microarray based method to selectively isolate clones containing species specific DNA. A particular preferred microarray embodiment of the present invention is a microarray comprising nucleic acid molecules encoding genes that are homologues of known genes or nucleic acid molecules that comprise genes or fragments thereof that elicit only limited or no matches to known genes. A further preferred microarray embodiment of the present invention is a microarray comprising nucleic acid molecules encoding genes or fragments thereof that are homologues of known genes and nucleic acid molecules that comprise genes or fragments thereof that elicit only limited or no matches to known genes. A further preferred microarray embodiment of the present invention is a microarray comprising nucleic acid molecules encoding genes or fragments thereof that elicit only limited or no matches to known genes.

It is understood that one or more of the molecules of the present invention, preferably one or more of the nucleic acid molecules or protein molecules or fragments thereof of the present invention may be utilized in a microarray based method. In a preferred embodiment of the present invention, one or more of the Glycine max nucleic acid molecules or protein molecules or fragments thereof of the present invention may be utilized in a microarray based method.

Nucleic acid molecules of the present invention may be used in site directed mutagenesis. Site-directed mutagenesis may be utilized to modify nucleic acid sequences, particularly as it is a technique that allows one or more of the amino acids encoded by a nucleic acid molecule to be altered (e.g. a threonine to be replaced by a methionine). Three basic methods for site-directed mutagenesis are often employed. These are cassette mutagenesis (Wells et al., Gene 34:315-23 (1985), the entirety of which is herein incorporated by reference), primer extension (Gilliam et al., Gene 12:129-137 (1980), the entirety of which is herein incorporated by reference); Zoller and Smith, Methods Enzymol. 100:468-500 (1983), the entirety of which is herein incorporated by reference; and Dalbadie-McFarland et al., Proc. Natl. Acad. Sci. (U.S.A.) 79:6409-6413 (1982), the entirety of which is herein incorporated by reference) and methods based upon PCR (Scharfet al., Science 233:1076-1078 (1986), the entirety of which is herein incorporated by reference; Higuchi et al., Nucleic Acids Res. 16:7351-7367 (1988), the entirety of which is herein incorporated by reference).

Any of the nucleic acid molecules of the present invention may either be modified by site-directed mutagenesis or used as, for example, nucleic acid molecules that are used to target other nucleic acid molecules for modification. It is understood that mutants with more than one altered nucleotide can be constructed using techniques that practitioners skilled in the art are familiar with such as isolating restriction fragments and ligating such fragments into an expression vector.

ApBACwich system has been developed to achieve site-directed integration of DNA into the genome. A 150 kb cotton BAC DNA is reported to have been transferred into a specific lox site in tobacco by biolistic bombardment and Cre-lox site specific recombination.

A construct or vector comprising a nucleic acid molecules of the present invention may be used in transformation. Exogenous genetic material may be transferred into a plant cell and the plant cell regenerated into a whole, fertile or sterile plant. Exogenous genetic material is any genetic material, whether naturally occurring or otherwise, from any source that is capable of being inserted into any organism. In a preferred embodiment of the present invention the exogenous genetic material can include Glycine max genetic material. Such genetic material may be transferred into either monocotyledons and dicotyledons including but not limited to the plants, Zea mays and Arabidopsis thaliana and soybean (See specifically, Chistou, Particle Bombardment for Genetic Engineering of Plants, pp. 63-69 (Zea mays), pp 50-60 (soybean), Biotechnology Intelligence Unit, Academic Press, San Diego, Calif. (1996), the entirety of which is herein incorporated by reference and generally Chistou, Particle Bombardment for Genetic Engineering of Plants, Biotechnology Intelligence Unit, Academic Press, San Diego, Calif. (1996), the entirety of which is herein incorporated by reference).

Transfer of a nucleic acid that encodes for a protein can result in overexpression of that protein in a transformed cell or transgenic plant. One or more of the proteins or fragments thereof encoded by nucleic acid molecules of the present invention may be overexpressed in a transformed cell or transformed plant. Such overexpression may be the result of transient or stable transfer of the exogenous material.

Exogenous genetic material may be transferred into a plant cell by the use of a DNA vector or construct designed for such a purpose. Vectors have been engineered for transformation of large DNA inserts into plant genomes. Vectors have been designed to replicate in both E. coli and A. tumefaciens and have all of the features required for transferring large inserts of DNA into plant chromosomes (Choi and Wing, at the website genome.clemson.edu/protocols2-nj.html July, 1998). ApBACwich system has been developed to achieve site-directed integration of DNA into the genome. A 150 kb cotton BAC DNA is reported to have been transferred into a specific lox site in tobacco by biolistic bombardment and Cre-lox site specific recombination.

A construct or vector may include a plant promoter to express the protein or protein fragment of choice. A number of promoters which are active in plant cells have been described in the literature. These include the nopaline synthase (NOS) promoter (Ebert et al., Proc. Natl. Acad. Sci. (U.S.A.) 84:5745-5749 (1987), the entirety of which is herein incorporated by reference), the octopine synthase (OCS) promoter (which are carried on tumor-inducing plasmids of Agrobacterium tumefaciens), the caulimovirus promoters such as the cauliflower mosaic virus (CaMV) 19S promoter (Lawton et al., Plant Mol. Biol. 9:315-324 (1987), the entirety of which is herein incorporated by reference) and the CAMV 35S promoter (Odell et al., Nature 313:810-812 (1985), the entirety of which is herein incorporated by reference), the figwort mosaic virus 35S-promoter, the light-inducible promoter from the small subunit of ribulose-1,5-bisphosphate carboxylase (ssRUBISCO), the Adh promoter (Walker et al., Proc. Natl. Acad. Sci. (U.S.A.) 84:6624-6628 (1987), the entirety of which is herein incorporated by reference), the sucrose synthase promoter (Yang et al., Proc. Natl. Acad. Sci. (U.S.A.) 87:4144-4148 (1990), the entirety of which is herein incorporated by reference), the R gene complex promoter (Chandler et al., The Plant Cell 1:1175-1183 (1989), the entirety of which is herein incorporated by reference), and the chlorophyll a/b binding protein gene promoter, etc. These promoters have been used to create DNA constructs which have been expressed in plants; see, e.g., PCT publication WO 84/02913, herein incorporated by reference in its entirety.

Promoters which are known or are found to cause transcription of DNA in plant cells can be used in the present invention. Such promoters may be obtained from a variety of sources such as plants and plant viruses. It is preferred that the particular promoter selected should be capable of causing sufficient expression to result in the production of an effective amount of protein to cause the desired phenotype. In addition to promoters which are known to cause transcription of DNA in plant cells, other promoters may be identified for use in the current invention by screening a plant cDNA library for genes which are selectively or preferably expressed in the target tissues or cells.

For the purpose of expression in source tissues of the plant, such as the leaf, seed, root or stem, it is preferred that the promoters utilized in the present invention have relatively high expression in these specific tissues. For this purpose, one may choose from a number of promoters for genes with tissue- or cell-specific or -enhanced expression. Examples of such promoters reported in the literature include the chloroplast glutamine synthetase GS2 promoter from pea (Edwards et al., Proc. Natl. Acad. Sci. (U.S.A.) 87:3459-3463 (1990), herein incorporated by reference in its entirety), the chloroplast fructose-1,6-biphosphatase (FBPase) promoter from wheat (Lloyd et al., Mol. Gen. Genet. 225:209-216 (1991), herein incorporated by reference in its entirety), the nuclear photosynthetic ST-LS1 promoter from potato (Stockhaus et al., EMBO J. 8:2445-2451 (1989), herein incorporated by reference in its entirety), the phenylalanine ammonia-lyase (PAL) promoter and the chalcone synthase (CHS) promoter from Arabidopsis thaliana. Also reported to be active in photosynthetically active tissues are the ribulose-1,5-bisphosphate carboxylase (RbcS) promoter from eastern larch (Larix laricina), the promoter for the cab gene, cab6, from pine (Yamamoto et al., Plant Cell Physiol. 35:773-778 (1994), herein incorporated by reference in its entirety), the promoter for the Cab-1 gene from wheat (Fejes et al., Plant Mol. Biol. 15:921-932 (1990), herein incorporated by reference in its entirety), the promoter for the CAB-1 gene from spinach (Lubberstedt et al., Plant Physiol. 104:997-1006 (1994), herein incorporated by reference in its entirety), the promoter for the cab1R gene from rice (Luan et al., Plant Cell. 4:971-981 (1992), the entirety of which is herein incorporated by reference), the pyruvate, orthophosphate dikinase (PPDK) promoter from Zea mays (Matsuoka et al., Proc. Natl. Acad. Sci. (U.S.A.) 90:9586-9590 (1993), herein incorporated by reference in its entirety), the promoter for the tobacco Lhcb1*2 gene (Cerdan et al., Plant Mol. Biol. 33:245-255. (1997), herein incorporated by reference in its entirety), the Arabidopsis thaliana SUC2 sucrose-H+ symporter promoter (Truernit et al., Planta. 196:564-570 (1995), herein incorporated by reference in its entirety), and the promoter for the thylacoid membrane proteins from spinach (psaD, psaF, psaE, PC, FNR, atpC, atpD, cab, rbcS). Other promoters for the chlorophyll a/b-binding proteins may also be utilized in the present invention, such as the promoters for LhcB gene and PsbP gene from white mustard (Sinapis alba; Kretsch et al., Plant Mol. Biol. 28:219-229 (1995), the entirety of which is herein incorporated by reference).

For the purpose of expression in sink tissues of the plant, such as the tuber of the potato plant, the fruit of tomato, or the seed of Zea mays, wheat, rice, and barley, it is preferred that the promoters utilized in the present invention have relatively high expression in these specific tissues. A number of promoters for genes with tuber-specific or -enhanced expression are known, including the class I patatin promoter (Bevan et al., EMBO J. 8:1899-1906 (1986); Jefferson et al., Plant Mol. Biol. 14995-1006 (1990), both of which are herein incorporated by reference in its entirety), the promoter for the potato tuber ADPGPP genes, both the large and small subunits, the sucrose synthase promoter (Salanoubat and Belliard, Gene. 60:47-56 (1987), Salanoubat and Belliard, Gene. 84:181-185 (1989), both of which are incorporated by reference in their entirety), the promoter for the major tuber proteins including the 22 kd protein complexes and proteinase inhibitors (Hannapel, Plant Physiol. 101:703-704 (1993), herein incorporated by reference in its entirety), the promoter for the granule bound starch synthase gene (GBSS) (Visser et al., Plant Mol. Biol. 17:691-699 (1991), herein incorporated by reference in its entirety), and other class I and II patatins promoters (Koster-Topfer et al., Mol. Gen. Genet. 219:390-396 (1989); Mignery et al., Gene. 62:27-44 (1988), both of which are herein incorporated by reference in their entirety).

Other promoters can also be used to express a fructose 1,6 bisphosphate aldolase gene in specific tissues, such as seeds or fruits. The promoter for β-conglycinin (Chen et al., Dev. Genet. 10:112-122 (1989), herein incorporated by reference in its entirety) or other seed-specific promoters such as the napin and phaseolin promoters, can be used. The zeins are a group of storage proteins found in Zea mays endosperm. Genomic clones for zein genes have been isolated (Pedersen et al., Cell 29:1015-1026 (1982), herein incorporated by reference in its entirety), and the promoters from these clones, including the 15 kD, 16 kD, 19 kD, 22 kD, 27 kD, and gamma genes, could also be used. Other promoters known to function, for example, in Zea mays, include the promoters for the following genes: waxy, Brittle, Shrunken 2, Branching enzymes I and II, starch synthases, debranching enzymes, oleosins, glutelins, and sucrose synthases. A particularly preferred promoter for Zea mays endosperm expression is the promoter for the glutelin gene from rice, more particularly the Osgt-1 promoter (Zheng et al., Mol. Cell Biol. 13:5829-5842 (1993), herein incorporated by reference in its entirety). Examples of promoters suitable for expression in wheat include those promoters for the ADPglucose pyrophosphorylase (ADPGPP) subunits, the granule bound and other starch synthases, the branching and debranching enzymes, the embryogenesis-abundant proteins, the gliadins, and the glutenins. Examples of such promoters in rice include those promoters for the ADPGPP subunits, the granule bound and other starch synthases, the branching enzymes, the debranching enzymes, sucrose synthases, and the glutelins. A particularly preferred promoter is the promoter for rice glutelin, Osgt-1. Examples of such promoters for barley include those for the ADPGPP subunits, the granule bound and other starch synthases, the branching enzymes, the debranching enzymes, sucrose synthases, the hordeins, the embryo globulins, and the aleurone specific proteins.

Root specific promoters may also be used. An example of such a promoter is the promoter for the acid chitinase gene (Samac et al., Plant Mol. Biol. 25:587-596 (1994), the entirety of which is herein incorporated by reference). Expression in root tissue could also be accomplished by utilizing the root specific subdomains of the CaMV35S promoter that have been identified (Lam et al., Proc. Natl. Acad. Sci. (U.S.A.) 86:7890-7894 (1989), herein incorporated by reference in its entirety). Other root cell specific promoters include those reported by Conkling et al. (Conkling et al., Plant Physiol. 93:1203-1211 (1990), the entirety of which is herein incorporated by reference).

Additional promoters that may be utilized are described, for example, in U.S. Pat. Nos. 5,378,619, 5,391,725, 5,428,147, 5,447,858, 5,608,144, 5,608,144, 5,614,399, 5,633,441, 5,633,435, and 4,633,436, all of which are herein incorporated in their entirety. In addition, a tissue specific enhancer may be used (Fromm et al., The Plant Cell 1:977-984 (1989), the entirety of which is herein incorporated by reference).

Constructs or vectors may also include, with the coding region of interest, a nucleic acid sequence that acts, in whole or in part, to terminate transcription of that region. For example, such sequences have been isolated including the Tr7 3′ sequence and the nos 3′ sequence (Ingelbrecht et al., The Plant Cell 1:671-680 (1989), the entirety of which is herein incorporated by reference; Bevan et al., Nucleic Acids Res. 11:369-385 (1983), the entirety of which is herein incorporated by reference), or the like.

A vector or construct may also include regulatory elements. Examples of such include the Adh intron 1 (Callis et al., Genes and Develop. 1:1183-1200 (1987), the entirety of which is herein incorporated by reference), the sucrose synthase intron (Vasil et al., Plant Physiol. 91:1575-1579 (1989), the entirety of which is herein incorporated by reference) and the TMV omega element (Gallie et al., The Plant Cell 1:301-311 (1989), the entirety of which is herein incorporated by reference). These and other regulatory elements may be included when appropriate.

A vector or construct may also include a selectable marker. Selectable markers may also be used to select for plants or plant cells that contain the exogenous genetic material. Examples of such include, but are not limited to, a neo gene (Potrykus et al., Mol. Gen. Genet. 199:183-188 (1985), the entirety of which is herein incorporated by reference) which codes for kanamycin resistance and can be selected for using kanamycin, G418, etc.; a bar gene which codes for bialaphos resistance; a mutant EPSP synthase gene (Hinchee et al., Bio/Technology 6:915-922 (1988), the entirety of which is herein incorporated by reference) which encodes glyphosate resistance; a nitrilase gene which confers resistance to bromoxynil (Stalker et al., J. Biol. Chem. 263:6310-6314 (1988), the entirety of which is herein incorporated by reference); a mutant acetolactate synthase gene (ALS) which confers imidazolinone or sulphonylurea resistance (European Patent Application 154,204 (Sept. 11, 1985), the entirety of which is herein incorporated by reference); and a methotrexate resistant DHFR gene (Thillet et al., J. Biol. Chem. 263:12500-12508 (1988), the entirety of which is herein incorporated by reference).

A vector or construct may also include a transit peptide. Incorporation of a suitable chloroplast transit peptide may also be employed (European Patent Application Publication Number 0218571, the entirety of which is herein incorporated by reference). Translational enhancers may also be incorporated as part of the vector DNA. DNA constructs could contain one or more 5′ non-translated leader sequences which may serve to enhance expression of the gene products from the resulting mRNA transcripts. Such sequences may be derived from the promoter selected to express the gene or can be specifically modified to increase translation of the mRNA. Such regions may also be obtained from viral RNAs, from suitable eukaryotic genes, or from a synthetic gene sequence. For a review of optimizing expression of transgenes, see Koziel et al., Plant Mol. Biol. 32:393-405 (1996), the entirety of which is herein incorporated by reference.

A vector or construct may also include a screenable marker. Screenable markers may be used to monitor expression. Exemplary screenable markers include a β-glucuronidase or uidA gene (GUS) which encodes an enzyme for which various chromogenic substrates are known (Jefferson, Plant Mol. Biol, Rep. 5:387-405 (1987), the entirety of which is herein incorporated by reference; Jefferson et al., EMBO J. 6:3901-3907 (1987), the entirety of which is herein incorporated by reference); an R-locus gene, which encodes a product that regulates the production of anthocyanin pigments (red color) in plant tissues ((Dellaporta et al., Stadler Symposium 11:263-282 (1988), the entirety of which is herein incorporated by reference); a β-lactamase gene (Sutcliffe et al., Proc. Natl. Acad. Sci. (U.S.A.) 75:3737-3741 (1978), the entirety of which is herein incorporated by reference), a gene which encodes an enzyme for which various chromogenic substrates are known (e.g., PADAC, a chromogenic cephalosporin); a luciferase gene (Ow et al., Science 234:856-859 (1986), the entirety of which is herein incorporated by reference) a xylE gene (Zukowsky et al., Proc. Natl. Acad. Sci. (U.S.A.) 80:1101-1105 (1983), the entirety of which is herein incorporated by reference) which encodes a catechol dioxygenase that can convert chromogenic catechols; an (x-amylase gene (Ikatu et al., Bio/Technol. 8:241-242 (1990), the entirety of which is herein incorporated by reference); a tyrosinase gene (Katz et al., J. Gen. Microbiol. 129:2703-2714 (1983), the entirety of which is herein incorporated by reference) which encodes an enzyme capable of oxidizing tyrosine to DOPA and dopaquinone which in turn condenses to melanin; an α-galactosidase, which will turn a chromogenic α-galactose substrate.

Included within the terms “selectable or screenable marker genes” are also genes which encode a secretable marker whose secretion can be detected as a means of identifying or selecting for transformed cells. Examples include markers which encode a secretable antigen that can be identified by antibody interaction, or even secretable enzymes which can be detected catalytically. Secretable proteins fall into a number of classes, including small, diffusible proteins detectable, e.g., by ELISA, small active enzymes detectable in extracellular solution (e.g., α-amylase, β-lactarnase, phosphinothricin transferase), or proteins which are inserted or trapped in the cell wall (such as proteins which include a leader sequence such as that found in the expression unit of extension or tobacco PR-S). Other possible selectable and/or screenable marker genes will be apparent to those of skill in the art.

Methods and compositions for transforming a bacteria and other microorganisms are known in the art (see for example Sambrook et al., Molecular Cloning: A Laboratory Manual, Second Edition, Cold Spring Harbor Laboratory Press, Cold Spring Harbor, N.Y., (1989), the entirety of which is herein incorporated by reference).

There are many methods for introducing transforming nucleic acid molecules into plant cells. Suitable methods are believed to include virtually any method by which nucleic acid molecules may be introduced into a cell, such as by Agrobacterium infection or direct delivery of nucleic acid molecules such as, for example, by PEG-mediated transformation, by electroporation or by acceleration of DNA coated particles, etc. (Pottykus, Ann. Rev. Plant Physiol. Plant Mol. Biol. 42:205-225 (1991), the entirety of which is herein incorporated by reference; Vasil, Plant Mol. Biol. 25:925-937 (1994), the entirety of which is herein incorporated by reference. For example, electroporation has been used to transform Zea mays protoplasts (Fromm et al., Nature 312:791-793 (1986), the entirety of which is herein incorporated by reference).

Technology for introduction of DNA into cells is well known to those of skill in the art. Four general methods for delivering a gene into cells have been described: (1) chemical methods (Graham and van der Eb, Virology, 54:536-539 (1973), the entirety of which is herein incorporated by reference); (2) physical methods such as microinjection (Capecchi, Cell 22:479-488 (1980), electroporation (Wong and Neumann, Biochem. Biophys. Res. Commun., 107:584-587 (1982); Fromm et al., Proc. Natl. Acad. Sci. (U.S.A.), 82:5824-5828 (1985); U.S. Pat. No. 5,384,253; and the gene gun (Johnston and Tang, Methods Cell Biol. 43:353-365 (1994), all of which the entirety is herein incorporated by reference; (3) viral vectors (Clapp, Clin. Perinatol., 20:155-168 (1993); Lu et al., J. Exp. Med., 178:2089-2096 (1993); Eglitis and Anderson, Biotechniques, 6:608-614 (1988), all of which the entirety is herein incorporated by reference); and (4) receptor-mediated mechanisms (Curiel et al., Hum. Gen. Ther., 3:147-154 (1992); Wagner et al., Proc. Natl. Acad. Sci. U.S.A., 89:6099-6103 (1992), all of which the entirety is herein incorporated by reference).

Acceleration methods that may be used include, for example, microprojectile bombardment and the like. One example of a method for delivering transforming nucleic acid molecules to plant cells is microprojectile bombardment. This method has been reviewed by Yang and Christou, eds., Particle Bombardment Technologyfor Gene Transfer, Oxford Press, Oxford, England (1994), the entirety of which is herein incorporated by reference). Non-biological particles (microprojectiles) that may be coated with nucleic acids and delivered into cells by a propelling force. Exemplary particles include those comprised of tungsten, gold, platinum, and the like.

A particular advantage of microprojectile bombardment, in addition to it being an effective means of reproducibly, and stably transforming monocotyledons, is that neither the isolation of protoplasts (Cristou et al., Plant Physiol. 87:671-674 (1988), the entirety of which is herein incorporated by reference) nor the susceptibility of Agrobacterium infection is required. An illustrative embodiment of a method for delivering DNA into maize cells by acceleration is a biolistics-particle delivery system, which can be used to propel particles coated with DNA through a screen, such as a stainless steel or Nytex screen, onto a filter surface covered with corn cells cultured in suspension. Gordon-Kamm et al., describes the basic procedure for coating tungsten particles with DNA (Gordon-Kamm et al., Plant Cell 2:603-618 (1990), the entirety of which is herein incorporated by reference). The screen disperses the tungsten nucleic acid particles so that they are not delivered to the recipient cells in large aggregates. A particle delivery system suitable for use with the present invention is the helium acceleration PDS-1000/He gun which is available from Bio-Rad Laboratories (Bio-Rad, Hercules, California) (Sanford et al., Technique 3:3-16 (1991), the entirety of which is herein incorporated by reference).

For the bombardment, cells in suspension may be concentrated on filters. Filters containing the cells to be bombarded are positioned at an appropriate distance below the microprojectile stopping plate. If desired, one or more screens are also positioned between the gun and the cells to be bombarded.

Alternatively, immature embryos or other target cells may be arranged on solid culture medium. The cells to be bombarded are positioned at an appropriate distance below the macroprojectile stopping plate. If desired, one or more screens are also positioned between the acceleration device and the cells to be bombarded. Through the use of techniques set forth herein one may obtain up to 1000 or more foci of cells transiently expressing a marker gene. The number of cells in a focus which express the exogenous gene product 48 hours post-bombardment often range from one to ten and average one to three.

In bombardment transformation, one may optimize the prebombardment culturing conditions and the bombardment parameters to yield the maximum numbers of stable transformants. Both the physical and biological parameters for bombardment are important in this technology. Physical factors are those that involve manipulating the DNA/microprojectile precipitate or those that affect the flight and velocity of either the macro- or microprojectiles. Biological factors include all steps involved in manipulation of cells before and immediately after bombardment, the osmotic adjustment of target cells to help alleviate the trauma associated with bombardment, and also the nature of the transforming DNA, such as linearized DNA or intact supercoiled plasmids. It is believed that pre-bombardment manipulations are especially important for successful transformation of immature embryos.

In another alternative embodiment, plastids can be stably transformed. Methods disclosed for plastid transformation in higher plants include particle gun delivery of DNA containing a selectable marker and targeting of the DNA to the plastid genome through homologous recombination (Svab et al. Proc. Natl. Acad. Sci. (U.S.A.) 87:8526-8530 (1990): Svab and Maliga Proc. Natl. Acad. Sci. (U.S.A.) 90:913-917 (1993)); (Staub, J. M. and Maliga, P. EMBO J. 12:601-606 (1993), U.S. Pat. Nos. 5, 451,513 and 5,545,818 all of which are herein incorporated by reference in their entirety).

Accordingly, it is contemplated that one may wish to adjust various aspects of the bombardment parameters in small scale studies to fully optimize the conditions. One may particularly wish to adjust physical parameters such as gap distance, flight distance, tissue distance, and helium pressure. One may also minimize the trauma reduction factors by modifying conditions which influence the physiological state of the recipient cells and which may therefore influence transformation and integration efficiencies. For example, the osmotic state, tissue hydration and the subculture stage or cell cycle of the recipient cells may be adjusted for optimum transformation. The execution of other routine adjustments will be known to those of skill in the art in light of the present disclosure.

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art. See, for example the methods described (Fraley et al., Biotechnology 3:629-635 (1985); Rogers et al., Meth. In Enzymol, 153:253-277 (1987), both of which are herein incorporated by reference in their entirety. Further, the integration of the Ti-DNA is a relatively precise process resulting in few rearrangements. The region of DNA to be transferred is defined by the border sequences, and intervening DNA is usually inserted into the plant genome as described (Spielmann et al., Mol. Gen. Genet., 205:34 (1986), the entirety of which is herein incorporated by reference).

Modern Agrobacterium transformation vectors are capable of replication in E. coli as well as Agrobacterium, allowing for convenient manipulations as described (Klee et al., In: Plant DNA Infectious Agents, T. Hohn and J. Schell, eds., Springer-Verlag, New York, pp. 179-203 (1985), the entirety of which is herein incorporated by reference. Moreover, recent technological advances in vectors for Agrobacterium-mediated gene transfer have improved the arrangement of genes and restriction sites in the vectors to facilitate construction of vectors capable of expressing various polypeptide coding genes. The vectors described have convenient multi-linker regions flanked by a promoter and a polyadenylation site for direct expression of inserted polypeptide coding genes and are suitable for present purposes (Rogers et al., Meth. In Enzymol., 153:253-277 (1987), the entirety of which is herein incorporated by reference). In addition, Agrobacterium containing both armed and disarmed Ti genes can be used for the transformations. In those plant strains where Agrobacterium-mediated transformation is efficient, it is the method of choice because of the facile and defined nature of the gene transfer.

A transgenic plant formed using Agrobacterium transformation methods typically contains a single gene on one chromosome. Such transgenic plants can be referred to as being heterozygous for the added gene. More preferred is a transgenic plant that is homozygous for the added structural gene; i.e., a transgenic plant that contains two added genes, one gene at the same locus on each chromosome of a chromosome pair. A homozygous transgenic plant can be obtained by sexually mating (selfing) an independent segregant transgenic plant that contains a single added gene, germinating some of the seed produced and analyzing the resulting plants produced for the gene of interest.

It is also to be understood that two different transgenic plants can also be mated to produce offspring that contain two independently segregating added, exogenous genes. Selfing of appropriate progeny can produce plants that are homozygous for both added, exogenous genes that encode a polypeptide of interest. Back-crossing to a parental plant and out-crossing with a non-transgenic plant are also contemplated, as is vegetative propagation.

Transformation of plant protoplasts can be achieved using methods based on calcium phosphate precipitation, polyethylene glycol treatment, electroporation, and combinations of these treatments. See for example (Potrykus et al., Mol. Gen. Genet., 205:193-200(1986); Lorzetal., Mol. Gen. Genet., 199:178, (1985); Frommetal., Nature, 319:791, (1986); Uchimiya et al., Mol. Gen. Genet. 204:204, (1986); Callis et al., Genes and Development, 1183,(1987); Marcotte et al., Nature, 335:454, (1988), all of which the entirety is herein incorporated by reference).

Application of these systems to different plant strains depends upon the ability to regenerate that particular plant strain from protoplasts. Illustrative methods for the regeneration of cereals from protoplasts are described (Fujimura et al., Plant Tissue Culture Letters, 2:74, (1985); Toriyama et al., Theor Appl. Genet. 205:34. (1986); Yamada et al., Plant Cell Rep., 4:85, (1986); Abdullah et al., Biotechnology, 4:1087, (1986), all of which the entirety is herein incorporated by reference).

To transform plant strains that cannot be successfully regenerated from protoplasts, other ways to introduce DNA into intact cells or tissues can be utilized. For example, regeneration of cereals from immature embryos or explants can be effected as described (Vasil, Biotechnology, 6:397, (1988), the entirety of which is herein incorporated by reference). In addition, “particle gun” or high-velocity microprojectile technology can be utilized (Vasil et al., Bio/Technology 10:667, (1992), the entirety of which is herein incorporated by reference).

Using the latter technology, DNA is carried through the cell wall and into the cytoplasm on the surface of small metal particles as described (Klein et al., Nature, 328:70, (1987); Klein et al., Proc. Natl. Acad. Sci. (U.S.A.), 85:8502-8505, (1988); McCabe et al., Biotechnology, 6:923, (1988), all of which the entirety is herein incorporated by reference). The metal particles penetrate through several layers of cells and thus allow the transformation of cells within tissue explants.

Other methods of cell transformation can also be used and include but are not limited to introduction of DNA into plants by direct DNA transfer into pollen (Zhou et al., Methods in Enzymology, 101:433, (1983); Hess et al., Intern Rev. Cytol., 107:367, (1987); Luo et al., Plant Mol. Biol. Reporter, 6:165, (1988), all of which the entirety is herein incorporated by reference), by direct injection of DNA into reproductive organs of a plant (Pena et al., Nature, 325:274, (1987), the entirety of which is herein incorporated by reference), or by direct injection of DNA into the cells of immature embryos followed by the rehydration of desiccated embryos (Neuhaus et al., Theor. Appl. Genet., 75:30, (1987), the entirety of which is herein incorporated by reference).

The regeneration, development, and cultivation of plants from single plant protoplast transformants or from various transformed explants is well known in the art (Weissbach and Weissbach, In: Methods for Plant Molecular Biology, (Eds.), Academic Press, Inc., San Diego, Calif., (1988), the entirety of which is herein incorporated by reference). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous gene that encodes a protein of interest is well known in the art. Preferably, the regenerated plants are self-pollinated to provide homozygous transgenic plants, as discussed before. Otherwise, pollen obtained from the regenerated plants is crossed to seed-grown plants of agronomically important lines. Conversely, pollen from plants of these important lines is used to pollinate regenerated plants. A transgenic plant of the present invention containing a desired polypeptide is cultivated using methods well known to one skilled in the art.

There are a variety of methods for the regeneration of plants from plant tissue. The particular method of regeneration will depend on the starting plant tissue and the particular plant species to be regenerated.

Methods for transforming dicots, primarily by use of Agrobacterium tumefaciens, and obtaining transgenic plants have been published for cotton (U.S. Pat. No. 5,004,863, U.S. Pat. No. 5,159,135, U.S. Pat. No. 5,518,908, all of which the entirety is herein incorporated by reference); soybean (U.S. Pat. No. 5,569,834, U.S. Pat. No. 5,416,011, McCabe et al., Biotechnology 6:923, (1988), Christou et al., Plant Physiol., 87:671-674 (1988), all of which the entirety is herein incorporated by reference); Brassica (U.S. Pat. No. 5,463,174, the entirety of which is herein incorporated by reference); peanut (Cheng et al., Plant Cell Rep. 15:653-657 (1996), McKently et al., Plant Cell Rep. 14:699-703 (1995), all of which the entirety is herein incorporated by reference); papaya (Yang et al., (1996), the entirety of which is herein incorporated by reference); pea (Grant et al., Plant Cell Rep. 15:254-258, (1995), the entirety of which is herein incorporated by reference).

Transformation of monocotyledons using electroporation, particle bombardment, and Agrobacterium have also been reported. Transformation and plant regeneration have been achieved in asparagus (Bytebier et al., Proc. Natl. Acad. Sci. (U.S.A.) 84:5345, (1987), the entirety of which is herein incorporated by reference); barley (Wan and Lemaux, Plant Physiol 104:37, (1994), the entirety of which is herein incorporated by reference); maize (Rhodes et al., Science 240:204, (1988), Gordon-Kamm et al., Plant Cell, 2:603, (1990), Fromm et al., Bio/Technology 8:833, (1990), Koziel et al., Bio/Technology 11:194, (1993), Armstrong et al., Crop Science 35:550-557, (1995), all of which the entirety is herein incorporated by reference); oat (Somers et al., Bio/Technology, 10:1589, (1992), the entirety of which is herein incorporated by reference); orchardgrass (Horn et al., Plant Cell Rep. 7:469, (1988), the entirety of which is herein incorporated by reference); rice (Toriyama et al., Theor Appl. Genet. 205:34, (1986); Park et al., Plant Mol. Biol., 32:1135-1148, (1996); Abedinia et al., Aust. J. Plant Physiol. 24:133-141, (1997); Zhang and Wu, Theor. Appl. Genet. 76:835, (1988); Zhang et al., Plant Cell Rep. 7:379, (1988); Battraw and Hall, Plant Sci. 86:191-202, (1992); Christou et al., Bio/Technology 9:957, (1991), all of which the entirety is herein incorporated by reference); sugarcane (Bower and Birch, Plant J. 2:409, (1992), the entirety of which is herein incorporated by reference); tall fescue (Wang et al., Bio/Technology 10:691, (1992), the entirety of which is herein incorporated by reference), and wheat (Vasil et al., Bio/Technology 10:667, (1992), the entirety of which is herein incorporated by reference; U.S. Pat. No. 5,631,152, the entirety of which is herein incorporated by reference.

Assays for gene expression based on the transient expression of cloned nucleic acid constructs have been developed by introducing the nucleic acid molecules into plant cells by polyethylene glycol treatment, electroporation, or particle bombardment (Marcotte, et al., Nature, 335:454-457 (1988), the entirety of which is herein incorporated by reference; Marcotte, et al., Plant Cell, 1:523-532 (1989), the entirety of which is herein incorporated by reference; McCarty, et al., Cell 66:895-905 (1991), the entirety of which is herein incorporated by reference; Hattori, et al., Genes Dev. 6:609-618 (1992), the entirety of which is herein incorporated by reference; Goff, et al., EMBO J. 9:2517-2522 (1990), the entirety of which is herein incorporated by reference). Transient expression systems may be used to functionally dissect gene constructs (See generally, Mailga et al., Methods in Plant Molecular Biology, Cold Spring Harbor Press (1995)).

Any of the nucleic acid molecules of the present invention may be introduced into a plant cell in a permanent or transient manner in combination with other genetic elements such as vectors, promoters enhancers etc. Further any of the nucleic acid molecules of the present invention may be introduced into a plant cell in a manner that allows for over expression of the protein or fragment thereof encoded by the nucleic acid molecule.

Nucleic acid molecules of the present invention may be used in cosuppression. Cosuppression is the reduction in expression levels, usually at the level of RNA, of a particular endogenous gene or gene family by the expression of a homologous sense construct that is capable of transcribing mRNA of the same strandedness as the transcript of the endogenous gene (Napoli et al., Plant Cell 2:279-289 (1990), the entirety of which is herein incorporated by reference; van der Krol et al., Plant Cell 2:291-299 (1990), the entirety of which is herein incorporated by reference). Cosuppression may result from stable transformation with a single copy nucleic acid molecule that is homologous to a nucleic acid sequence found with the cell (Prolls and Meyer, Plant J. 2:465-475 (1992), the entirety of which is herein incorporated by reference) or with multiple copies of a nucleic acid molecule that is homologous to a nucleic acid sequence found with the cell (Mittlesten et al:, Mol. Gen. Genet. 244: 325-330 (1994), the entirety of which is herein incorporated by reference). Genes, even though different, linked to homologous promoters may result in the cosuppression of the linked genes (Vaucheret, CR. Acad. Sci. III 316: 1471-1483 (1993), the entirety of which is herein incorporated by reference).

This technique has, for example been applied to generate white flowers from red petunia and tomatoes that do not ripen on the vine. Up to 50% of petunia transformants that contained a sense copy of the chalcone synthase (CHS) gene produced white flowers or floral sectors; this was as a result of the post-transcriptional loss of mRNA encoding CHS (Flavell, Proc. Natl. Acad. Sci. (U.S.A.) 91:3490-3496 (1994)), the entirety of which is herein incorporated by reference). Cosuppression may require the coordinate transcription of the transgene and the endogenous gene, and can be reset by a developmental control mechanism (Jorgensen, Trends Biotechnol, 8:340344 (1990), the entirety of which is herein incorporated by reference; Meins and Kunz, In: Gene Inactivation and Homologous Recombination in Plants (Paszkowski, J., ed.), pp. 335-348. Kluwer Academic, Netherlands (1994), the entirety of which is herein incorporated by reference).

It is understood that one or more of the nucleic acids of the present invention comprising SEQ ID NO:1 or complement thereof through SEQ ID NO:304905 or complement thereof, may be introduced into a plant cell and transcribed using an appropriate promoter with such transcription resulting in the co-suppression of an endogenous protein.

Nucleic acid molecules of the present invention may be used to reduce gene function. Antisense approaches are a way of preventing or reducing gene function by targeting the genetic material (Mol et al., FEBS Lett. 268:427-430 (1990), the entirety of which is herein incorporated by reference). The objective of the antisense approach is to use a sequence complementary to the target gene to block its expression and create a mutant cell line or organism in which the level of a single chosen protein is selectively reduced or abolished. Antisense techniques have several advantages over other ‘reverse genetic’ approaches. The site of inactivation and its developmental effect can be manipulated by the choice of promoter for antisense genes or by the timing of external application or microinjection. Antisense can manipulate its specificity by selecting either unique regions of the target gene or regions where it shares homology to other related genes (Hiatt et al., In Genetic Engineering, Setlow (ed.), Vol. 11, New York: Plenum 49-63 (1989), the entirety of which is herein incorporated by reference).

The principle of regulation by antisense RNA is that RNA that is complementary to the target mRNA is introduced into cells, resulting in specific RNA:RNA duplexes being formed by base pairing between the antisense substrate and the target mRNA (Green et al., Annu. Rev. Biochem. 55:569-597 (1986), the entirety of which is herein incorporated by reference). Under one embodiment, the process involves the introduction and expression of an antisense gene sequence. Such a sequence is one in which part or all of the normal gene sequences are placed under a promoter in inverted orientation so that the ‘wrong’ or complementary strand is transcribed into a noncoding antisense RNA that hybridizes with the target mRNA and interferes with its expression (Takayama and Inouye, Crit. Rev. Biochem. Mol. Biol. 25:155-184 (1990), the entirety of which is herein incorporated by reference). An antisense vector is constructed by standard procedures and introduced into cells by transformation, transfection, electroporation, microinjection, or by infection, etc. The type of transformation and choice of vector will determine whether expression is transient or stable. The promoter used for the antisense gene may influence the level, timing, tissue, specificity, or inducibility of the antisense inhibition.

It is understood that protein synthesis activity in a plant cell may be reduced or depressed by growing a transformed plant cell containing a nucleic acid molecule of the present invention.

Antibodies have been expressed in plants (Hiatt et al., Nature 342:76-78 (1989), the entirety of which is herein incorporated by reference; Conrad and Fielder, Plant Mol. Biol. 26:1023-1030 (1994), the entirety of which is herein incorporated by reference). Cytoplasmic expression of a scFv (single-chain Fv antibodies) has been reported to delay infection by artichoke mottled crinkle virus. Transgenic plants that express antibodies directed against endogenous proteins may exhibit a physiological effect (Philips et al., EMBO J. 16:4489-4496 (1997), the entirety of which is herein incorporated by reference; Marion-Poll, Trends in Plant Science 2:447-448 (1997), the entirety of which is herein incorporated by reference). For example, expressed anti-abscisic antibodies reportedly result in a general perturbation of seed development (Philips et al., EMBO J. 16:4489-4496 (1997)).

Nucleic acid molecules of the present invention may be used as antibodies. Antibodies that are catalytic may also be expressed in plants (abzymes). The principle behind abzymes is that since antibodies may be raised against many molecules, this recognition ability can be directed toward generating antibodies that bind transition states to force a chemical reaction forward (Persidas, Nature Biotechnology 15:1313-1315 (1997), the entirety of which is herein incorporated by reference; Baca et al., Ann. Rev. Biophys. Biomol. Struct. 26:461-493 (1997), the entirety of which is herein incorporated by reference). The catalytic abilities of abzymes may be enhanced by site directed mutagenesis. Examples of abzymes are, for example, set forth in U.S. Pat. No. 5,658,753; U.S. Pat. No. 5,632,990; U.S. Pat. No. 5,631,137; U.S. Pat. No. 5,602,015; U.S. Pat. No. 5,559,538; U.S. Pat. No. 5,576,174; U.S. Pat. No. 5,500,358; U.S. Pat. No. 5,318,897; U.S. Pat. No. 5,298,409; U.S. Pat. No. 5,258,289 and U.S. Pat. No. 5,194,585, all of which are herein incorporated in their entirety.

It is understood that any of the antibodies of the present invention may be expressed in plants and that such expression can result in a physiological effect. It is also understood that any of the expressed antibodies may be catalytic.

In addition to the above discussed procedures, practitioners are familiar with the standard resource materials which describe specific conditions and procedures for the construction, manipulation and isolation of macromolecules (e.g., DNA molecules, plasmids, etc.), generation of recombinant organisms and the screening and isolating of clones, (see for example, Sambrook et al., Molecular Cloning: A Laboratory Manual, Cold Spring Harbor Press (1989); Mailga et al., Methods in Plant Molecular Biology, Cold Spring Harbor Press (1995), the entirety of which is herein incorporated by reference; Birren et al., Genome Analysis: Analyzing DNA, 1, Cold Spring Harbor, N.Y., the entirety of which is herein incorporated by reference).

Computer Media

One or more of the nucleotide sequence provided in SEQ ID NO: 1 through SEQ ID NO: 304905 or complements thereof can be “provided” in a variety of media to facilitate use. Such a medium can also provide a subset thereof in a form that allows a skilled artisan to examine the sequences. be recorded on computer readable media. As used herein, “computer readable media” refers to any medium that can be read and accessed directly by a computer. Such media include, but are not limited to: magnetic storage media, such as floppy discs, hard disc, storage medium, and magnetic tape: optical storage media such as CD-ROM; electrical storage media such as RAM and ROM; and hybrids of these categories such as magnetic/optical storage media. A skilled artisan can readily appreciate how any of the presently known computer readable mediums can be used to create a manufacture comprising computer readable medium having recorded thereon a nucleotide sequence of the present invention.

As used herein, “recorded” refers to a process for storing information on computer readable medium. A skilled artisan can readily adopt any of the presently known methods for recording information on computer readable medium to generate media comprising the nucleotide sequence information of the present invention. A variety of data storage structures are available to a skilled artisan for creating a computer readable medium having recorded thereon a nucleotide sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect and Microsoft Word, or represented in the form of an ASCII file, stored in a database application, such as DB2, Sybase, Oracle, or the like. A skilled artisan can readily adapt any number of data processor structuring formats (e.g., text file or database) in order to obtain computer readable medium having recorded thereon the nucleotide sequence information of the present invention.

By providing one or more of nucleotide sequences of the present invention, a skilled artisan can routinely access the sequence information for a variety of purposes. Computer software is publicly available which allows a skilled artisan to access sequence information provided in a computer readable medium. The examples which follow demonstrate how software which implements the BLAST (Altschul et al., J. Mol. Biol. 215:403-410 (1990)) and BLAZE (Brutlag et al., Comp. Chem. 17:203-207 (1993), the entirety of which is herein incorporated by reference) search algorithms on a Sybase system can be used to identify open reading frames (ORFs) within the genome that contain homology to ORFs or proteins from other organisms. Such ORFs are protein-encoding fragments within the sequences of the present invention and are useful in producing commercially important proteins such as enzymes used in amino acid biosynthesis, metabolism, transcription, translation, RNA processing, nucleic acid and a protein degradation, protein modification, and DNA replication, restriction, modification, recombination, and repair.

The present invention further provides systems, particularly computer-based systems, which contain the sequence information described herein. Such systems are designed to identify commercially important fragments of the nucleic acid molecule of the present invention. As used herein, “a computer-based system” refers to the hardware means, software means, and data storage means used to analyze the nucleotide sequence information of the present invention. The minimum hardware means of the computer-based systems of the present invention comprises a central processing unit (CPU), input means, output means, and data storage means. A skilled artisan can readily appreciate that any one of the currently available computer-based system are suitable for use in the present invention.

As indicated above, the computer-based systems of the present invention comprise a data storage means having stored therein a nucleotide sequence of the present invention and the necessary hardware means and software means for supporting and implementing a search means. As used herein, “data storage means” refers to memory that can store nucleotide sequence information of the present invention, or a memory access means which can access manufactures having recorded thereon the nucleotide sequence information of the present invention. As used herein, “search means” refers to one or more programs which are implemented on the computer-based system to compare a target sequence or target structural motif with the sequence information stored within the data storage means. Search means are used to identify fragments or regions of the sequence of the present invention that match a particular target sequence or target motif. A variety of known algorithms are disclosed publicly and a variety of commercially available software for conducting search means are available and can be used in the computer-based systems of the present invention. Examples of such software include, but are not limited to, MacPattern (EMBL), BLASTIN and BLASTIX (NCBIA). One of the available algorithms or implementing software packages for conducting homology searches can be adapted for use in the present computer-based systems.

The most preferred sequence length of a target sequence is from about 10 to 100 amino acids or from about 30 to 300 nucleotide residues. However, it is well recognized that during searches for commercially important fragments of the nucleic acid molecules of the present invention, such as sequence fragments involved in gene expression and protein processing, may be of shorter length.

As used herein, “a target structural motif,” or “target motif,” refers to any rationally selected sequence or combination of sequences in which the sequence(s) are chosen based on a three-dimensional configuration which is formed upon the folding of the target motif. There are a variety of target motifs known in the art. Protein target motifs include, but are not limited to, enzymatic active sites and signal sequences. Nucleic acid target motifs include, but are not limited to, promoter sequences, cis elements, hairpin structures and inducible expression elements (protein binding sequences).

Thus, the present invention further provides an input means for receiving a target sequence, a data storage means for storing the target sequences of the present invention sequence identified using a search means as described above, and an output means for outputting the identified homologous sequences. A variety of structural formats for the input and output means can be used to input and output information in the computer-based systems of the present invention. A preferred format for an output means ranks fragments of the sequence of the present invention by varying degrees of homology to the target sequence or target motif. Such presentation provides a skilled artisan with a ranking of sequences which contain various amounts of the target sequence or target motif and identifies the degree of homology contained in the identified fragment.

A variety of comparing means can be used to compare a target sequence or target motif with the data storage means to identify sequence fragments sequence of the present invention. For example, implementing software which implement the BLAST and BLAZE algorithms (Altschul et al., J. Mol. Biol. 215:403-410 (1990)) can be used to identify open frames within the nucleic acid molecules of the present invention. A skilled artisan can readily recognize that any one of the publicly available homology search programs can be used as the search means for the computer-based systems of the present invention.

Having now generally described the invention, the same will be more readily understood through reference to the following examples which are provided by way of illustration, and are not intended to be limiting of the present invention, unless specified.

### EXAMPLE 1

BACs are stable, non-chimeric cloning systems having genomic fragment inserts (100-300 kb) and their DNA can be prepared for most types of experiments including DNA sequencing. BAC vector, pBeloBAC11, is derived from the endogenous E. coli F-factor plasmid, which contains genes for strict copy number control and unidirectional origin of DNA replication. Additionally, pBeloBAC11 has three unique restriction enzyme sites (Hind III, Bam HI and Sph 1) located within the LacZ gene which can be used as cloning sites for megabase-size plant DNA. Indigo, another BAC vector contains Hind III and Eco RI cloning sites. This vector also contains a random mutation in the LacZ gene that allows for darker blue colonies.

As an alternative, the P1-derived artificial chromosome (PAC) can be used as a large DNA fragment cloning vector (Ioannou, et al., Nature Genet. 6:84-89 (1994), the entirety of which is herein incorporated by reference; Suzuki, et al., Gene 199:133-137 (1997), the entirety of which is herein incorporated by reference). The PAC vector has most of the features of the BAC system, but also contains some of the elements of the bacteriophage P1 cloning system.

BAC libraries are generated by ligating size-selected restriction digested DNA with pBeloBAC11 followed by electroporation into E. coli. BAC library construction and characterization is extremely efficient when compared to YAC (yeast artificial chromosome) library construction and analysis, particularly because of the chimerism associated with YACs and difficulties associated with extracting YAC DNA.

There are two general methods for preparing megabase-size DNA from plants. The protoplast method yields megabase-size DNA of high quality with minimal breakage.

The process involves preparing young leaves which are manually feathered with a razor-blade before being incubated for four to five hours with cell-wall-degrading enzymes.

The second method developed by Zhange et al., Plant J. 7:175-184 (1995) the entirety of which is herein incorporated by reference is a universal nuclei method that works well for several divergent plant taxa. Fresh or frozen tissue is homogenized with a blender or mortar and pestle. Nuclei are then isolated and embedded. DNA is prepared by the nucleic method often more concentrated and is reported to contain lower amounts of chloroplast DNA than the protoplast method.

Once protoplasts or nuclei are produced, they are embedded in an agarose matrix as plugs or microbeads. The agarose provides a support matrix to prevent shearing of the DNA while allowing enzymes and buffers to diffuse into the DNA. The DNA is purified and manipulated in the agarose and is stable for more than one year at 4° C.

Once high molecular weight DNA has been prepared, it is fragmented to the desired size range. In general, DNA fragmentation utilizes two general approaches, 1) physical shearing and 2) partial digestion with a restriction enzyme that cuts relatively frequently within the genome. Since physical shearing is not dependent upon the frequency and distribution of particular restriction enzymes sites, this method should yield the most random distribution of DNA fragments. However, the ends of the sheared DNA fragments must be repaired and cloned directly or restriction enzyme sites added by the addition of synthetic linkers. Because of the subsequent steps required to clone DNA fragmented by shearing, most protocols fragment DNA by partial restriction enzyme digestion. The advantage of partial restriction enzyme digestion is that no further enzymatic modification of the ends of the restriction fragments are necessary. Four common techniques that can be used to achieve reproducible partial digestion of megabase-size DNA are 1) varying the concentration of the restriction enzyme, 2) varying the time of incubation with the restriction enzyme 3) varying the concentration of an enzyme cofactor (e.g., Mg²⁺) and 4) varying the ratio of endonuclease to methylase.

There are three cloning sites in pBeloBAC 11, but only Hind III and Bam HI produce 5′ overhangs for easy vector dephosphorylation. These two restriction enzymes are primarily used to construct BAC libraries. The optimal partial digestion conditions for megabase-size DNA are determined by wide and narrow window digestions. To optimize the optimum amount of Hind III, 1, 2, 3, 10, and 5-units of enzyme are each added to 50 ml aliquots of microbeads and incubated at 37° C. for 20 minutes

After partial digestion of megabase-size DNA, the DNA is run on a pulsed-field gel, and DNA in a size range of 100-500 kb is excised from the gel. This DNA is ligated to the BAC vector or subjected to a second size selection on a pulsed field gel under different running conditions. Studies have previously reported that two rounds of size selection can eliminate small DNA fragments co-migrating with the selected range in the first pulse-field fractionation. Such a strategy results in an increase in insert sizes and a more uniform insert size distribution. A practical approach to performing size selections is to first test for the number of clones/microliter of ligation and insert size from the first size selected material. If the numbers are good (500 to 2000 white colony/microliter of ligation) and the size range is also good (50 to 300 kb) then a second size selection is practical. When performing a second size selection one expects a 80 to 95% decrease in the number of recombinant clones per transformation.

Twenty to two hundred nanograms of the size-selected DNA is ligated to dephosphorylated BAC vector (molar ratio of 10 to 1 in BAC vector excess). Most BAC libraries use a molar ratio of 5 to 15:1 (size selected DNA:BAC vector).

Transformation is carried out by electroporation and the transformation efficiency for BACs is about 40 to 1,500 transformants from one microliter of ligation product or 20 to 1000 transformants/ng DNA.

Several tests can be carried out to determine the quality of a BAC library. Three basic tests to evaluate the quality include: the genome coverage of a BAC library-average insert size, average number of clones hybridizing with single copy probes and chloroplast DNA content.

The determination of the average insert size of the library is assessed in two ways. First, during library construction every ligation is tested to determine the average insert size by assaying 20-50 BAC clones per ligation. DNA is isolated from recombinant clones using a standard mini preparation protocol, digested with Not I to free the insert from the BAC vector and then sized using pulsed field gel electrophoresis (Maule, Molecular Biotechnology 9:107-126 (1998), the entirety of which is herein incorporated by reference).

To determine the genome coverage of the library, it is screened with single copy RFLP markers distributed randomly across the genome by hybridization. Microtiter plates containing BAC clones are spotted onto Hybond membranes. Bacteria from 48 or 72 plates are spotted twice onto one membrane resulting in 18,000 to 27,648 unique clones on each membrane in either a 4×4 or 5×5 orientation. Since each clone is present twice, false positives are easily eliminated and true positives are easily recognized and identified.

Finally, the chloroplast DNA content in the BAC library is estimated by hybridizing three chloroplast genes spaced evenly across the chloroplast genome to the library on high density hybridization filters.

There are strategies for isolating rare sequences within the genome. For example, higher plant genomes can range in size from 100 Mb/1C (Arabidopsis) to 15,966 Mb/C (Triticum aestivum), (Arumuganathan and Earle, Plant Mol Bio Rep. 9:208-219 (1991), the entirety of which is herein incorporated by reference). The number of clones required to achieve a given probability that any DNA sequence will be represented in a genomic library is N=(ln(1−P))/(ln(1−L/G)) where N is the number of clones required, P is the probability desired to get the target sequence, L is the length of the average clone insert in base pairs and G is the haploid genome length in base pairs (Clarke et al., Cell 9:91-100 (1976) the entirety of which is herein incorporated by reference).

The soybean BAC library of the present invention is constructed in the pBeloBAC11 or similar vector. Inserts are generated by partial Eco RI or other enzymatic digestion of DNA from the cultivar A3244. The library provides approximately twenty fold coverage of the soybean genome.

### EXAMPLE 2

Two basic methods can be used for DNA sequencing, the chain termination method of Sanger et al., Proc. Natl. Acad. Sci. (U.S.A.) 74:5463-5467 (1977), the entirety of which is herein incorporated by reference and the chemical degradation method of Maxam and Gilbert, Proc. Natl. Acad. Sci. (U.S.A.) 74:560-564 (1977), the entirety of which is herein incorporated by reference. Automation and advances in technology such as the replacement of radioisotopes with fluorescence-based sequencing have reduced the effort required to sequence DNA (Craxton, Methods, 2:20-26 (1991), the entirety of which is herein incorporated by reference; Ju et al., Proc. Natl. Acad. Sci. (U.S.A.) 92:4347-4351 (1995), the entirety of which is herein incorporated by reference; Tabor and Richardson, Proc. Natl. Acad. Sci. (U.S.A.) 92:6339-6343 (1995), the entirety of which is herein incorporated by reference). Automated sequencers are available from, for example, Pharmacia Biotech, Inc., Piscataway, N.J. (Pharmacia ALF), LI-COR, Inc., Lincoln, Nebr. (LI-COR 4,000) and Millipore, Bedford, Mass. (Millipore BaseStation).

In addition, advances in capillary gel electrophoresis have also reduced the effort required to sequence DNA and such advances provide a rapid high resolution approach for sequencing DNA samples (Swerdlow and Gesteland, Nucleic Acids Res. 18:1415-1419 (1990); Smith, Nature 349:812-813 (1991); Luckey et al., Methods Enzymol. 218:154-172 (1993); Lu et al., J. Chromatog. A. 680:497-501 (1994); Carson et al., Anal. Chem. 65:3219-3226 (1993); Huang et al., Anal. Chem. 64:2149-2154 (1992); Kheterpal et al., Electrophoresis 17:1852-1859 (1996); Quesada and Zhang, Electrophoresis 17:1841-1851 (1996); Baba, Yakugaku Zasshi 117:265-281 (1997), all of which are herein incorporated by reference in their entirety).

A number of sequencing techniques are known in the art, including fluorescence-based sequencing methodologies. These methods have the detection, automation and instrumentation capability necessary for the analysis of large volumes of sequence data. Currently, the 377 DNA Sequencer (Perkin-Elmer Corp., Applied Biosystems Div., Foster City, Calif.) allows the most rapid electrophoresis and data collection. With these types of automated systems, fluorescent dye-labeled sequence reaction products are detected and data entered directly into the computer, producing a chromatogram that is subsequently viewed, stored, and analyzed using the corresponding software programs. These methods are known to those of skill in the art and have been described and reviewed (Birren et al., Genome Analysis: Analyzing DNA, 1, Cold Spring Harbor, N.Y., the entirety of which is herein incorporated by reference).

297 forward STCs are resequenced and the complex repeats within the these sequences are located. These forward STCS are designated: GM_{—}667_B2_H12_MF_{—}50, GM_{—}667_B2_H12_MF_{—}4BDT, GM_{—}667_B2_H12_MF_{—}40, GM_{—}667_B2_H11_MF_{—}50, GM_{—}667_B2_H11_MF_{—}4BDT, GM_{—}667_B2_H11_MF_{—}40, GM_{—}667_B2_H10_MF_{—}50, GM_{—}667_B2_H10_MF_{—}4BDT, GM_{—}667_B2_H10_MF_{—}40, GM_{—}667_B2_H09_MF_{—}35, GM_{—}667_B2_H08_MF_{—}50, GM_{—}667_B2_H08_MF_{—}40, GM_{—}667_B2_H08_MF_{—}35, GM_{—}667_B2_H07_MF_{—}4BDT, GM_{—}667_B2_H07_MF_{—}40, GM_{—}667_B2_H06_MF_{—}40, GM_{—}667_B2_H05_MF_{—}40, GM_{—}667_B2_H05_MF_{—}35, GM_{—}667_B2_H04_MF_{—}50, GM_{—}667_B2_H04_MF_{—}4BDT, GM_{—}667_B2_H04_MF_{—}40, GM_{—}667_B2_H03_MF_{—}40, GM_{—}667_B2_H02_MF_{—}50, GM_{—}667_B2_H02_MF_{—}4BDT, GM_{—}667_B2_H02_MF_{—}40, GM_{—}667_B2_H02_MF_{—}35, GM_{—}667_B2_H01_MF_{—}40, GM_{—}667_B2_H01_MF_{—}35, GM_{—}667_B2_G12_MF_{—}50, GM_{—}667_B2_G12_MF_{—}40, GM_{—}667_B2_G11_MF_{—}40, GM_{—}667_B2_G10_MF_{—}50, GM_{—}667_B2_G10_MF_{—}4BDT, GM_{—}667_B2_G10_MF_{—}35, GM_{—}667_B2_G09_MF_{—}4BDT, GM_{—}667_B2_G09_MF_{—}40, GM_{—}667_B2_G09_MF_{—}35, GM_{—}667_B2_G08_MF_{—}50, GM_{—}667_B2_G08_MF_{—}4BDT, GM_{—}667_B2_G08_MF_{—}40, GM_{—}667_B2_G07_MF_{—}4BDT, GM_{—}667_B2_G07_MF_{—}35, GM_{—}667_B2_G06_MF_{—}40, GM_{—}667_B2_G05_MF_{—}4BDT, GM_{—}667_B2_G05_MF_{—}40, GM_{—}667_B2_G05_MF_{—}35, GM_{—}667_B2_G04_MF_{—}50, GM_{—}667_B2_G04_MF_{—}4BDT, GM_{—}667_B2_G04_MF_{—}40, GM_{—}667_B2_G03_MF_{—}40, GM_{—}667_B2_G02_MF_{—}50, GM_{—}667_B2_G02_MF_{—}4BDT, GM_{—}667_B2_G02_MF_{—}40, GM_{—}667_B2_G02_MF_{—}35, GM_{—}667_B2_G01_MF_{—}4BDT, GM_{—}667_B2_G01_MF_{—}40, GM_{—}667_B2_G01_MF_{—}35, GM_{—}667_B2_F11_MF_{—}50, GM_{—}667_B2_F11_MF_{—}4BDT, GM_{—}667_B2_F11_MF_{—}40, GM_{—}667_B2_F10_MF_{—}4BDT, GM_{—}667_B2_F10_MF_{—}40, GM_{—}667_B2_F10MF_{—}35, GM_{—}667_B2_F09_MF_{—}50, GM_{—}667_B2_F09_MF_{—}4BDT, GM_{—}667_B2_F09_MF_{—}40, GM_{—}667_B2_F08_MF_{—}50, GM_{—}667_B2_F08_MF_{—}40, GM_{—}667_B2_F07_MF_{—}50, GM_{—}667_B2_F07_MF_{—}4BDT, GM_{—}667_B2_F07_MF_{—}40, GM_{—}667_B2_F07_MF_{—}35, GM_{—}667_B2_F06_MF_{—}40, GM_{—}667_B2_F05_MF_{—}4BDT, GM_{—}667_B2_F05_MF_{—}40, GM_{—}667_B2_F04_MF_{—}50, GM_{—}667_B2_F04_MF_{—}4BDT, GM_{—}667_B2_F04_MF_{—}40, GM_{—}667_B2_F04_MF_{—}35, GM_{—}667_B2_F03_MF_{—}40, GM_{—}667_B2_F02_MF_{—}4BDT, GM_{—}667_B2_F02_MF_{—}35, GM_{—}667_B2_F01_MF_{—}35, GM_{—}667_B2_E12_MF_{—}50, GM_{—}667_B2_E12_MF_{—}4BDT, GM_{—}667_B2_E12_MF_{—}40, GM_{—}667_B2_E10_MF_{—}50, GM_{—}667_B2_E10_MF_{—}4BDT, GM_{—}667_B2_E10_MF_{—}40, GM_{—}667_B2_E09_MF_{—}50, GM_{—}667_B2_E09_MF_{—}4BDT, GM_{—}667_B2_E09_MF_{—}40, GM_{—}667_B2_E08_MF_{—}40, GM_{—}667_B2_E07_MF_{—}4BDT, GM_{—}667_B2_E07_MF_{—}40, GM_{—}667_B2_E07_MF_{—}35, GM_{—}667_B2_E06_MF_{—}50, GM_{—}667_B2_E06_MF_{—}40, GM_{—}667_B2_E05_MF_{—}50, GM_{—}667_B2_E05_MF_{—}40, GM_{—}667_B2_E04_MF_{—}4BDT, GM_{—}667_B2_E04_MF_{—}40, GM_{—}667_B2_E03_MF_{—}4BDT, GM_{—}667_B2_E03_MF_{—}40, GM_{—}667_B2_E03_MF_{—}35, GM_{—}667_B2_E02_MF_{—}50, GM_{—}667_B2_E02_MF_{—}4BDT, GM_{—}667_B2_E02_MF_{—}40, GM_{—}667_B2_E02_MF_{—}35, GM_{—}667_B2_E01_MF_{—}4BDT, GM_{—}667_B2_E01_MF_{—}40, GM_{—}667_B2_E01_MF_{—}35, GM_{—}667_B2_D12_MF_{—}4BDT, GM_{—}667_B2_D12_MF_{—}40, GM_{—}667_B2_D11_MF_{—}50, GM_{—}667_B2_D11_MF_{—}4BDT, GM_{—}667_B2_D11MF_{—}40, GM_{—}667_B2_D11_MF_{—}35, GM_{—}667_B2_D10_MF_{—}50, GM_{—}667_B2_D10_MF_{—}40, GM_{—}667_B2_D10_MF_{—}35, GM_{—}667_B2_D09_MF_{—}4BDT, GM_{—}667_B2_D09_MF_{—}40, GM_{—}667_B2_D08_MF_{—}40, GM_{—}667_B2_D06_MF_{—}4BDT, GM_{—}667_B2_D06_MF_{—}40, GM_{—}667_B2_D06_MF_{—}35, GM_{—}667_B2_D05_MF_{—}50, GM_{—}667_B2_D05_MF_{—}40, GM_{—}667_B2_D04_MF_{—}50, GM_{—}667_B2_D04_MF_{—}4BDT, GM_{—}667_B2_D04_MF_{—}40, GM_{—}667_B2_D04_MF_{—}35, GM_{—}667_B2_D03_MF_{—}40, GM_{—}667_B2_D03_MF_{—}35, GM_{—}667_B2_D02_MF_{—}4BDT, GM_{—}667_B2_D02_MF_{—}40, GM_{—}667_B2_D02_MF_{—}35, GM_{—}667_B2_D01_MF_{—}40, GM_{—}667_B2_D01_MF_{—}35, GM_{—}667_B2_C12_MF_{—}50, GM_{—}667_B2_C12_MF_{—}4BDT, GM_{—}667_B2_C12_MF_{—}40, GM_{—}667_B2_C11_MF_{—}40, GM_{—}667_B2_C10_MF_{—}40, GM_{—}667_B2_C09_MF_{—}50, GM_{—}667_B2_C09_MF_{—}4BDT, GM_{—}667_B2_C09_MF_{—}40, GM_{—}667_B2_C08_MF_{—}50, GM_{—}667_B2_C08_MF_{—}40, GM_{—}667_B2_C07_MF_{—}4BDT, GM_{—}667_B2_C07_MF_{—}40, GM_{—}667_B2_C07_MF_{—}35, GM_{—}667_B2_C06_MF_{—}50, GM_{—}667_B2_C06_MF_{—}40, GM_{—}667_B2_C05_MF_{—}50, GM_{—}667_B2_C05_MF_{—}40, GM_{—}667_B2_C04_MF_{—}4BDT, GM_{—}667_B2_C04_MF_{—}40, GM_{—}667_B2_C04_MF_{—}35, GM_{—}667_B2_C03_MF_{—}35, GM_{—}667_B2_C02_MF_{—}40, GM_{—}667_B2_C02_MF_{—}35, GM_{—}667_B2_C01_MF_{—}40, GM_{—}667_B2_C01_MF_{—}35, GM_{—}667_B2_B12_MF_{—}4BDT, GM_{—}667_B2_B12_MF_{—}40, GM_{—}667_B2_B11_MF_{—}4BDT, GM_{—}667_B2_B11_MF_{—}40, GM_{—}667_B21B11_MF_{—}35, GM_{—}667_B2_B10_MF_{—}50, GM_{—}667_B2_B10_MF_{—}40, GM_{—}667_B2_B09_MF_{—}50, GM_{—}667_B2_B09_MF_{—}4BDT, GM_{—}667_B2_B09_MF_{—}40, GM_{—}667_B2_B07_MF_{—}4BDT, GM_{—}667_B2_B07_MF_{—}40, GM_{—}667_B2_B07_MF_{—}35, GM_{—}667_B2_B06_MF_{—}40, GM_{—}667_B2_B05_MF_{—}50, GM_{—}667_B2_B05_MF_{—}4BDT, GM_{—}667_B2_B05_MF_{—}40, GM_{—}667_B2_B04_MF_{—}35, GM_{—}667_B2_B03_MF_{—}40, GM_{—}667_B2_B03_MF_{—}35, GM_{—}667_B2_B02_MF_{—}4BDT, GM_{—}667_B2_B02_MF_{—}40, GM_{—}667_B2_B02_MF_{—}35, GM_{—}667_B2_A12_MF_{—}4BDT, GM_{—}667_B2_A12_MF_{—}40, GM_{—}667_B2_A11_MF_{—}4BDT, GM_{—}667_B2_A11_MF_{—}40, GM_{—}667_B2_A11_MF_{—}35, GM_{—}667_B2_A10_MF_{—}40, GM_{—}667_B2_A10_MF_{—}35, GM_{—}667_B2_A09_MF_{—}50, GM_{—}667_B2_A09_MF_{—}40, GM_{—}667_B2_A07_MF_{—}50, GM_{—}667_B2_A07_MF_{—}4BDT, GM_{—}667_B2_A07_MF_{—}40, GM_{—}667_B2_A07_MF_{—}35, GM_{—}667_B2_A06_MF_{—}40, GM_{—}667_B2_A06_MF_{—}35, GM_{—}667_B2_A05_MF_{—}50, GM_{—}667_B2_A05_MF_{—}4BDT, GM_{—}667_B2_A05_MF_{—}40, GM_{—}667_B2_A04_MF_{—}50, GM_{—}667_B2_A04_MF_{—}4BDT, GGM_{—}667_B2_A03_MF_{—}40, GM_{—}57_B2_C12_T7, GM_{—}57_B2_A0_T7, GM_{—}57_B2_A03_T7, GM_{—}57_B2_A05_T7, GM_{—}57_B2_A08_T7, GM_{—}57_B2_A09_T7, GM_{—}57_B2_A10_T7, GM_{—}57_B2_A12_T7, GM_{—}57_B2_B01_T7, GM_{—}57_B2_B02_T7, GM_{—}57_B2_B03_T7, GM_{—}57_B2_B04_T7, GM_{—}57_B2_B05_T7, GM_{—}57_B2_B06_T7, GM_{—}57_B2_B07_T7, GM_{—}57_B2_B08_T7, GM_{—}57_B2_B09_T7, GM_{—}57_B2_B11_T7, GM_{—}57_B2_B12_T7, GM_{—}57_B2_C01_T7, GM_{—}57_B2_C02_T7, GM_{—}57_B2_C03_T7, GM_{—}57_B2_C04_T7, GM_{—}57_B2_C05_T7, GM_{—}57_B2_C07_T7, GM_{—}57_B2_C08_T7, GM_{—}57_B2_C10_T7, GM_{—}57_B2_D01_T7, GM_{—}57_B2_D02_T7, GM_{—}57_B2_D03_T7, GM_{—}57_B2_D04_T7, GM_{—}57_B2_D05_T7, GM_{—}57_B2_D06_T7, GM_{—}57_B2_D07_T7, GM_{—}57_B2_D08_T7, GM_{—}57_B2_D10_T7, GM_{—}57_B2_D11_T7, GM_{—}57_B2_D12_T7, GM_{—}57_B2_E01_T7, GM_{—}57_B2_E02_T7, GM_{—}57_B2_E03_T7, GM_{—}57_B2_E04_T7, GM_{—}57_B2_E05_T7, GM_{—}57_B2_E06_T7, GM_{—}57_B2_E07_T7, GM_{—}57_B2_E08_T7, GM_{—}57_B2_E10_T7, GM_{—}57_B2_E11_T7, GM_{—}57_B2_E12_T7, GM_{—}57_B2_F01_T7, GM_{—}57_B2_F02_T7, GM_{—}57_B2_F03_T7, GM_{—}57_B2_F04_T7, GM_{—}57_B2_F05_T7, GM_{—}57_B2_F08_T7, GM_{—}57_B2_F09_T7, GM_{—}57_B2_F11_T7, GM_{—}57_B2_F12_T7, GM_{—}57_B2_G01_T7, GM_{—}57_B2_G02_T7, GM_{—}57_B2_G03_T7, GM_{—}57_B2_G04_T7, GM_{—}57_B2_G05_T7, GM_{—}57_B2_G06_T7, GM_{—}57_B2_G07_T7, GM_{—}57_B2_G08_T7, GM_{—}57_B2_G09_T7, GM_{—}57_B2_G10_T7, GM_{—}57_B2_G12_T7, GM_{—}57_B2_H01_T7, GM_{—}57_B2_H02_T7, GM_{—}57_B2_H03_T7, GM_{—}57_B2_H04_T7, GM_{—}57_B2_H05_T7, GM_{—}57_B2_H06_T7, GM_{—}57_B2_H08_T7, GM_{—}57_B2_H11_T7, GM_{—}57_B2_H12_T7, GM_{—}707_A2_D05_T7, GM_{—}707_A2_D08_T7, GM_{—}707_A2_D12_T7, GM_{—}707_A2_F08_T7, GM_{—}707_A2_G01_T7, GM_{—}707_A2_G06_T7, GM_{—}707_A2_G08_T7, GM_{—}707_A2_G11_T7, GM_{—}707_A2_H06_T7, GM_{—}707_A2_H07_T7. The clones names corresponding to these STCs are determined by truncation before the “_{—T}7” or the first “_{—MF”.}

### EXAMPLE 3

To identify sequences containing microsatellites or simple sequence repeats (SSR), a SSR repeat pattern library is generated by using a Perl program, SSR_{—generator.pl, developed at Monsanto. The library contains repeat patterns of di-, tri-, tetra-, penta- and hexa- nucleotide repeats, a total of} 5421 patterns. The length of di-, tri-, tetra-, penta- and hexa- nucleotide repeat units were 18, 12, 9, 5 and 4, respectively. These repeat patterns are used to search against the BAC-end sequence databases by the BLASTN program. If the search is performed on both strands, complementary and replicated patterns of an SSR library are removed from the library to avoid redundancy of SSRs. For di-nucleotide repeats, there are four unique patterns, i.e. (CA)n, (CT)n, (CG)n and (AT)n. Product scores are used as a criteria to extract potential SSRs from BAC-ends. If a product score is equal or greater than 90, the sequences are further examined.

The SSR-containing sequences identified from BAC ends are searched against each other as well as the existing SSR collections by using BLASTN, and clustering of the sequences is performed by using CLUSTER2, a tool developed at Monsanto. The minimal match-length is set to 100 base pairs. Any redundant sequences are removed and the unique ones are then passed through a visible inspection to further remove those with not enough flanking sequences for primer design and those with substantial ambiguous nucleotides.

Primers are designed from good quality unique sequences. A public available primer design software program, PRIMER 3, (Cambridge, Mass.) is used. PRIMER 3 can be accessed though the internet at genome.wi.mit.edu/cgi-bin/primer/primer3.cgi. Default parameters are used except those for product size and primer size are changed. Product Size is Min: 80, Opt: 100, Max: 120, while Primer Size is Min: 18, Opt: 22 and Max: 27. Oligos are synthesized by Genosis Biotechnologies, Inc (Houston, Tex.).

The above protocols are used to develop primers from Sequence id GM_M02_A2_B07_MR_MR containing the following nucleotide composition:

AGGCGTTTTNCCTTGATACCTTCGNAGGTCCANCCTTTTNCTTGCTGTAT
CGACTCATTAACACCAAGCTCGGTGAGCACTCTGAAGATTATGACAACTT
TCGNTGATCTTTTTGTCATCGATATTNTAGNAGAGACCAATCTTTCTTCT
TCAAATGTCGCTCATGATATTTATTGTAATTATCTTCAATGTATGTCCAA
AAAGTTAACCTTTTTTGGACCCCCACAATAGAAATCTTTGAAATATTTAG
CCATGTGTTGGCAAGCCATTCATATTTCTTTGCGGAGAAACATGATCTAT
TGTGTCTTTCGGATGCTTCTTCTATGTcttcttcttcttcttcttcttct
tcttcttcttCATTGACCACAATATTATCCAACTCAACTTAGGTGCAAAA
TGGTGGAATTTGAGACTTTGACGCANAGTCAGATGGTGCGTCATGCTCTT
TCATTACATTGGACATCATNTACTACCCTTTGAAGACCCTCGATCCATGG
AAGGGTTAATTGGTG

This sequence contains CTT dinucleotide repeats with a repeat unit of 11. Using the Primer 3 program, two primers are selected: SER157F GTGTCTTTCGGATGCTTCTTCT and SER157R CACCATTTTGCACCTAAGTTGA. When these two primers are used to amplify genomic DNAs from eight different varieties, Minsoy, Noir, PIC, HS-1, A3244, H6686, A0868 and H5088, three alleles are detected. Sizes of these alleles ranged from 80 to 110 bp The size variation in the PCR products result from repeat numbers in different varieties.

PCR Reaction Conditions

Genomic DNA is isolated from young leaves of Glycine max or Glycine soja plants. Two leaf discs are collected (approximately 40 mg) from a healthy leaf and stored on wet ice or at 4° C. Tissue samples are then freeze-dried and stored at −20° C. or −80° C. The frozen samples are kept as dry as possible and sealed from contact with the atmosphere. The freeze-dried samples from −20° C. or −80° C., are allowed to warm up to room temperature prior to unsealing or opening. One leaflet (or 2 leaf discs) is inserted into an 1.5 ml Eppendorf tube, placed on dry ice, and crushed with a wooden dowel. Approximately 200 μl of microprep buffer (25 ml extraction buffer (350 mM sorbitol, 100 mM Tris-base, 5 mM EDTA-Na₂), 25 ml nuclei lysis buffer (1M Tris/HCl, 0.5 M EDTA, 5 M NaCl, 2% CTAB), 10 ml 5% sarkosyl, 0.1 g Na bisulfite) is added to each sample. The sample is then homogenized. An additional 550 μl of microprep buffer is added, vortexed for about 30-60 seconds, and incubated at 65° C. for about 60 minutes. About 700 μl chloroform/isoamyl (24:1) is added, mixed well for about 10-30 seconds. Centrifugation of the tubes is performed at approximately 10,000 rpm for 5 minutes in a microcentrifuge. The aqueous phase is transferred into a new tube and RNA is removed from the extract by the addition of 30 μl of RNase (10 mg/ml) to the aqueous phase and incubated for 1 hour at room temperature. Approximately 500 μl ice-cold isopropanol is added to the aqueous extract, and the tubes inverted until the DNA precipitated. The precipitated solution is kept at 4° C. for about 1 hour or overnight. Centrifugation of the tubes is performed at approximately 10,000 rpm for 5 minutes in a microcentrifuge. The supernatant is discarded and the pellet washed 1-3 times with 200 μl 70% ethanol. The ethanol is removed using a micropipette and pellet dried at 37° C. for 10 minutes. The DNA is dissolved in 50 μl TE (10 mM Tris-HCl pH8.0, 0.1 mM EDTA), then kept overnight at 4° C. Centrifugation of the tubes is performed at approximately 10,000 rpm for 5 minutes and then the supernatant is transferred into new tubes. Using this method, approximately 2 μg of DNA per mg of fresh leaf tissue is extracted.

DNA concentration is measured by a Spectrometry (Molecular Devices, Sunnyvale, Calif.) and adjusted to proper concentration for use as template. The total volume for PCR reaction is 20 ul. The reaction mixture contains: Template DNA at a concentration of 15 ng, 0.15 uM of primer, 0.03 unit of Taq DNA polymerase (Perkin Elmer), 50 uM of dNTP, the Reaction buffer contains, 10 mM Tris.HCl pH8.5,1.5 mM MgCl2, 50 mM KCl and water is added to a total volume of 20 ul.

The PCR is performed on a Perkin Elmer DNA Thermal Cycler 9700 using the following cycle profile: hold at 94° C. for 3 min, 32 cycles of 94° C. for 25 second, 47° C. for 25 second and 72° C. for 25 second, and 72° C. for 3min of final extension.

An acrylamide gel is prepared using 56.5 ml water, 3.5 ml 10×TAE buffer, 10.5 ml 40 acrylamide stock solution, 50 μl TEMED, 0.06 g ammonium persulfate. To each PCR, sample 20 μl of formamide loading dye is added to each sample and the samples are denatured at 90° C. for 3 minutes with a 4° C. hold in a thermocycler. 1.5 μl of each sample is loaded onto the gel. Gels are run at constant wattage to give a constant heat development during electrophoresis at 40 to 50 Volt/cm of the gel length. Gels should be run at approximately 50° C. during electrophoresis. Electrophoresis is stopped when the Bromophenol blue dye is at the bottom of the gel. After electrophoresis, the gel is stained in 1×SYBR solution for 15 to 20 minutes with vigorous shaking. A Gel image is recorded using an Alpha-InnoTech imager.

### EXAMPLE 4

In order to create a file containing complex repeats, the GCG (Madison, Wis.) REPEAT program is used to determine initial internal repeats. Stringency is defined as 19 matched bases out of every contiguous 20 bases in the repeated diagonals part of the REPEAT program algorithm. After the REPEAT program is run on the STCs, a REPEAT output filed is processed with the UNIX utilities grep, sort, uniq and sed to produce a GCG pattern file. The GCG pattern file is broken into size groups: <20, 20-39, 40-59, 60-79, 80-89, 100-119, 120-139, 140-159, 160-179, 180-199, 200-219, and >220. Each pattern group is compared against the entire STC library or subset thereof using the GCG FINDPATTERNS program. Sequences of size 1-19 are allowed no mismatches. The 20-39 group are allowed one mismatch. A pattern of size n is allowed floor (n/20) mismatches. Patterns that occurred in at least 100 STCs are selected in this step. The results of the FINDPATTERNS program is post-processed with the UNIX utilities grep, sort and uniq and with the GCG REFORMAT program to produce GCG sequence files. Each sequence file is derived from a selected pattern and placed in a subdirectory that corresponds to its size group. GELSTART, GELENTER, GELMERGE and GELASSEMBLE are used to coalesce similar sequences of each size group. Patterns are 90% similar before they are aligned and the patterns overlap by at least two thirds of the modal length in their length group. The GELSTART program creates a subdirectory which contains the individual and the coalesced consensus sequences. The consensus sequences are placed into a single directory and a FASTA style sequence library is constructed from it. The REPEAT-MASKER program is used to mask the original STCs. The unmasked sequences that remain afterward are concatenated into 100 KB pseudo-sequences. The pseudo-sequences are fed back into this algorithm and the new repeat patterns that result are added to the repeat library. The algorithm is iterated 3 times.

The repeat library is compared to the STCs using NCBI BLASTN version 2.0. HSPs are reported if they satisfied the criteria of:
“observed fractional match” >=“allowed fractional match” where “observed fractional match” is defined as:
“fraction of HSP similarity”×“fraction of query sequence in HSP” and “allowed fractional match” is defined as:
(“repeat length”−“floor (repeat length/20)”)/“repeat length”

Alternatively, the repeat library is compared to the STCs by an algorithm that is written in the C programming language and is compiled with optimization. Using a repeat library patterns file containing 3,302 complex repeat sequences from Glycine max, 304905 Glycine max STC sequences are searched and repeat coordinates are identified in these sequences.

STC and repeat library DNA sequences are represented by the characters A, C, G, and T. Ambiguous sequence characters allow for combinations of these characters, as defined by the IUPAC-IUB (the Wisconsin Package version 10.0, Genetics Computer Group, Madison, Wis.). For example, A or T is represented by W, G or C by S, and A or C or G or T by N. DNA sequence characters are represented as 4 binary digits (bits), where 0001 represents A, 0010 represents C, 0100 represents G, and 1000 represents T. Using standard Boolean logic, A or T (W) is equivalent to applying the logical OR operator to 0001 and 1000, the result being 1001. The table below shows all standard symbols and their computer representation for this method.

| IUPAC-IUB | | Computer |
|---|---|---|
| Symbol | Meaning | Representation |
| A | A | 0001 |
| C | C | 0010 |
| G | G | 0100 |
| T | T | 1000 |
| K | G or T (Keto) | 1100 |
| M | A or C (aMino) | 0011 |
| R | A or G (puRine) | 0101 |
| S | G or C (Strong pairing) | 0110 |
| W | A or T (Weak pairing) | 1001 |
| Y | C or T (pYrimidine) | 1010 |
| B | C or G or T (not A) | 1110 |
| D | A or G or T (not C) | 1101 |
| H | A or C or T (not G) | 1011 |
| V | A or C or G (not T or U) | 0111 |
| N | A or C or G or T | 1111 |

When matching sequence patterns, a match occurs only when a symbol in the sequence being searched is a subset of the symbol appearing in the pattern. For example, an A in the pattern will match only an A in the sequence, whereas an R in the pattern will match any of A, G, or R (but no other symbols). The AND operator is applied to the computer representation of the pattern symbol and the sequence symbol, and a match occurs if the result is identical to the sequence symbol. For example, A matches A because 0001 (pattern) AND 0001 (sequence) equals 0001 (result), and the result equals the sequence. An R in the pattern matches an A in the sequence because 0101 (pattern) AND 0001 (sequence) equals 0001 (result), and the result equals the sequence. An S in the pattern does not match an A in the sequence: 0110 (pattern) AND 0001 (sequence) equals 0000 (result), the result not matching the sequence. Using this algorithm, pattern matching becomes a byte by byte comparison using the AND operator.

The algorithm allows the user to define the number of mismatches as a fraction of the number of characters in the pattern. For example, a 5% mismatch frequency allows for one mismatch every 20 pattern characters. This works out to 0 mismatches for a pattern of 1-19 characters, 1 mismatch for a pattern of 20-39 characters, 2 mismatches for a pattern of 40-59 characters, and so on.

The searching algorithm aligns the pattern sequence with the DNA sequence at every possible position on both DNA strands and counts the number of mismatches in the alignment. If the number of mismatches is less than or equal to the number permitted, then a match is recorded.

The patterns and the DNA sequence are stored in Fasta-format DNA sequence files. The length of the patterns and the DNA sequences are limited only by available computer memory. The computer program first loads the patterns into memory. Each DNA sequence is then loaded sequentially from the Fasta file and searched sequentially with each pattern, allowing for the mismatch frequency designated by the user. The reverse complement of the DNA sequence is generated and again searched with the patterns. The coordinates of the pattern matches for each sequence and the name of the pattern that matched are saved in memory. Once a sequence has been searched with all patterns, the coordinates of the patterns are sorted into order, and the name of the DNA sequence, the name of the pattern, and the coordinates of the match are written to an output file.

We claim:

1. A substantially purified nucleic acid molecule, said nucleic acid molecule capable of specifically hybridizing to a second nucleic acid molecule having a nucleic acid sequence selected from the group consisting of SEQ ID NO: 1 through SEQ ID NO: 304905 or complement or fragment thereof.

2. The substantially purified nucleic acid molecule according to claim 1, wherein said nucleic acid molecule comprises a microsatellite sequence.

3. The substantially purified nucleic acid molecule according to claim 1, wherein said nucleic acid molecule comprises a region having a single nucleotide polymorphism.

4. The substantially purified nucleic acid molecule according to claim 1, wherein said nucleic acid molecule comprises a nucleic acid molecule having a nucleic acid sequence selected from the group consisting of SEQ ID NO: 1 through SEQ ID NO: 304905 or complement thereof.

5. The substantially purified nucleic acid molecule according to claim 4, wherein said nucleic acid molecule further comprises a bacterial ORI site.

6. The substantially purified nucleic acid molecule according to claim 1, wherein said nucleic acid molecule has a promoter or partial promoter region.

7. The substantially purified nucleic acid molecule according to claim 6, wherein said promoter region comprises a CAAT cis element and a TATA cis element and an additional cis element.

8. A substantially purified nucleic acid molecule comprising a nucleic acid molecule or fragment thereof having a pair of defined ends, wherein said pair of defined ends are selected from the defined ends in Table A.

9. The substantially purified nucleic acid molecule according to claim 8, wherein said molecule comprises a nucleic acid molecule having one or two of said defined ends.

10. The substantially purified nucleic acid molecule according to claim 9, wherein said molecule comprises a nucleic acid molecule having two of said defined ends.

11. A substantially purified protein or fragment thereof encoded by a first nucleic acid molecule which specifically hybridizes to a second nucleic acid molecule, said second nucleic acid molecule having a nucleic acid sequence selected from the group consisting of SEQ ID NO:1 through SEQ ID NO:304905 or complements thereof.

12. A transformed plant having a nucleic acid molecule which comprises:
(A) an exogenous promoter region which functions in a plant cell to cause the production of a mRNA molecule; which is linked to
(B) a structural nucleic acid molecule, wherein said structural nucleic acid molecule is selected from the group consisting of SEQ ID NO:1 through SEQ ID NO:304905 or complements thereof or fragment of either; which is linked to
(C) a 3′ non-translated sequence that functions in a plant cell to cause termination of transcription and addition of polyadenylated ribonucleotides to a 3′ end of said mRNA molecule.

13. The transformed plant according to claim 12, wherein said structural nucleic acid molecule is in the antisense orientation.

14. The transformed plant according to claim 12, wherein said plant is a dicot.

15. The transformed plant according to claim 12, wherein said plant is a monocot.
